(19) United States
(12) Patent Application Publication
(10) Pub. No.: US 2026/0148859 A1
(43) Pub. Date: May 28, 2026

(54) METHODS AND SYSTEMS FOR PREDICTING A CUTANEOUS PRIMARY DISEASE SITE

(71) Applicant: Foundation Medicine, Inc., Boston, MA (US)

(72) Inventors: Douglas A. MATA, Cambridge, MA (US); Brennan DECKER, Winchester, MA (US); Ethan S. SOKOL, Somerville, MA (US); Zoe R. FLEISCHMANN, Jamaica Plain, MA (US)

(73) Assignee: Foundation Medicine, Inc., Boston, MA (US)

(21) Appl. No.: 19/121,583

(22) PCT Filed: Oct. 16, 2023
(86) PCT No.: PCT/US2023/076959

§ 371 Date: Apr. 16, 2025

Related U.S. Application Data
(60) Provisional application No. 63/416,716, filed on Oct. 17, 2022.

(51) Int. Cl. G16H 50/50; G16B 20/00; G16B 40/20

(52) U.S. Cl. CPC G16H 50/50; G16B 20/00; G16B 40/20

(57) ABSTRACT

Methods for predicting a cutaneous primary site of disease are described. The methods may comprise, for example, receiving, using one or more processors, sequence read data associated with a sample from the individual, selecting, using the one or more processors, a plurality of reads from the sequence read data, determining, using the one or more processors, an ultra-violet (UV) signature metric based on the selected plurality of reads, inputting, using the one or more processors, the UV signature metric into a statistical model, and predicting, using the one or more processors, the primary site of the disease in the individual based on an output of the statistical model.

### CROSS-REFERENCE TO RELATED APPLICATIONS

This application claims the priority benefit of U.S. Provisional Patent Application Ser. No. 63/416,716, filed Oct. 17, 2022, the contents of which are incorporated herein by reference in their entirety.

### FIELD OF THE DISCLOSURE

The present disclosure relates generally to methods and systems for analyzing genomic profiling data, and more specifically to methods and systems for predicting a primary site of a disease using genomic profiling data.

### BACKGROUND

Conventional histopathological examination with immunohistochemistry may be used to identify a primary site (e.g., location of an origin) of a disease (e.g., cancer) based on a sample. However, histopathological examination may be unable to identify some diseases and will classify these samples as associated with a cancer of an unknown primary site. For example, histopathological examination performs poorly in identifying cutaneous origin in a subset of cancers of an unknown primary site. This is particularly the case with metastatic squamous cell carcinomas and melanomas. As such, histopathological examination may result in incorrect diagnoses.

For example, if an individual presents to a healthcare provider as experiencing symptoms such as chest pain and is coughing up blood, the healthcare provider may perform an x-ray on the individual's lungs. If the x-ray shows the presence of tumors in the lung, the healthcare provider may take a tissue sample of the lung. Based on an analysis of the tissue sample of the lung, the healthcare provider may identify the lungs as the primary site or origin for the carcinoma. However, in some instances, the tumor may correspond to a metastatic disease that has a cutaneous origin. This misdiagnosis can lead to the application of incorrect therapies and treatment being administered to the individual. For example, the healthcare provider, believing the disease originated in the lungs, may recommend removing the tumor as a potential cure. But if the disease is cutaneous in origin, removing the tumor from the lung will not cure the individual as the disease originated elsewhere in the individual. In this case, where the disease is cutaneous in origin, treatment options such as immunotherapy may be more successful. Accordingly, there is a need to supplement conventional histopathological diagnoses of disease to confirm or refute the origin of a disease.

### BRIEF SUMMARY

Disclosed herein are methods and systems for determining whether the primary site of a disease (e.g., cancer) in an individual is cutaneous in origin. For example, one or more embodiments of the present disclosure may relate to using one or more UV signatures (e.g., COSMIC UV mutational signatures) to determine a cancer is cutaneous in origin.

UV radiation exposure causes a characteristic mutational pattern associated with elevated tumor mutational burden (TMB) via the formation of pyrimidine-pyrimidine photodimers (COSMIC signature 7). COSMIC single base substitution signature 7 is highly specific to UV-mediated mutagenesis, suggests cutaneous origin in cancers of uncertain primary site, and may also flag potential misdiagnoses by conventional histopathology. See e.g., Signatures of Mutational Processes in Human Cancer, COSMIC—Catalogue of Somatic Mutations in Cancer, https://cancer.sanger.ac.uk/signatures/signatures v2/; Mata D A, et. al., Prevalence of UV Mutational Signatures Among Cutaneous Primary Tumors, JAMA Network Open, 2022. doi:10.1001/jamanetworkopen.2022.3833. The inventors of this disclosure developed a method that leverages UV signatures in conjunction with other predictive features (e.g., the presence of specific genomic alterations and/or the co-occurrence of other clinicopathologic features) to predict cutaneous origin in cancers of uncertain primary site and to identify misdiagnoses in cancers erroneously thought to arise from other primary sites.

Embodiments of the present disclosure may be further used to overturn incorrectly rendered diagnoses. Additionally, embodiments of the present disclosure may be used to correctly identify a stage of a cancer. For example, a one-centimeter tumor identified in the lungs, where the primary site of the disease corresponds to the lungs may be determined to be a stage one cancer. However, the same tumor identified in the lungs, where the primary site of the disease may correspond to a different site, may be determined to be a stage four cancer, e.g., because the disease has metastasized. Correctly staging a disease may have important prognosis and treatment implications. For example, for a stage one cancer in the lungs, removal of the tumor may be an effective treatment option, while for a stage four cancer of cutaneous origin, immunotherapy may be an effective treatment option. Thus, embodiments of the present disclosure can address some of the limitations of conventional histopathology by providing another metric for consideration by healthcare providers when determining a diagnosis.

Embodiments of the present disclosure comprise systems and methods for determining whether a disease in an individual is associated with a cutaneous primary site. In one or more examples, the methods can include: providing a plurality of nucleic acid molecules obtained from a sample from a subject, ligating one or more adapters onto one or more nucleic acid molecules from the plurality of nucleic acid molecules, amplifying the one or more ligated nucleic acid molecules from the plurality of nucleic acid molecules, capturing amplified nucleic acid molecules from the amplified nucleic acid molecules, sequencing, by a sequencer, the captured nucleic acid molecules to obtain a plurality of sequence reads that represent the captured nucleic acid molecules, receiving, at one or more processors, sequence read data for the plurality of sequence reads, determining, using the one or more processors, an ultra-violet (UV) signature metric based on the selected plurality of reads, inputting, using the one or more processors, the UV signature metric into a statistical model, and predicting, using the one or more processors, the primary site of the disease in the individual based on an output of the statistical model.

In some examples of this disclosure, the output is indicative of a cutaneous primary site of the disease or a non-cutaneous primary site of the disease. In some examples of this disclosure, the UV signature metric comprises a binary value indicative of a UV signature call and a confidence score associated with the UV signature call. In some examples of this disclosure, determining the binary value and confidence score is based on a fit of a predetermined number of short variants to a UV reference signature. In some examples of this disclosure, the predetermined number of short variants corresponds to three or more variants.

In some examples of this disclosure, the UV signature metric is associated with a catalogue of somatic mutations in one or more of cancer (COSMIC) single base substitution (SBS) signature 7a, signature 7b, signature 7c, signature 7d, or a combination thereof. In some examples, the one or more mutational signatures comprise a COSMIC doublet base substitution (DBS) signature 1. In some examples of this disclosure, the method further comprises determining, using the one or more processors, whether a UV signature corresponding to the UV signature metric was detected in the sample.

In some examples of this disclosure, the method further comprises determining, using the one or more processors, genomic features of the selected plurality of reads, biomarker features of the selected plurality of reads, or a combination thereof. In some examples, the method further comprises inputting the genomic features of the selected plurality of reads, biomarker features of the selected plurality of reads, or a combination thereof into the statistical model to predict the primary site.

In some examples of this disclosure, the method further comprises determining, using the one or more processors, genomic features of the selected plurality of reads, biomarker features of the selected plurality of reads, or a combination thereof. In some examples, the method further comprises inputting the genomic features of the selected plurality of reads, biomarker features of the selected plurality of reads, or a combination thereof into the statistical model to predict the primary site. In some examples, the genomic features comprise the presence of one or more predetermined short variants or an absence of the one or more predetermined short variants.

In one or more examples of this disclosure, the subject is suspected of having or is determined to have cancer. In one or more examples of the embodiment described above, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pincaloma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypercosinophilia, chronic eosinophilic leukemia, neuroendocrine cancers, or a carcinoid tumor.

In one or more examples of the embodiment described above, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In one or more examples of this disclosure, the method further comprises treating the subject with an anti-cancer therapy. In one or more examples of this disclosure, the anti-cancer therapy comprises a targeted anti-cancer therapy. In one or more examples of this disclosure, the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane 1131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligco), moxctumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In one or more examples of this disclosure, the method further comprises obtaining the plurality of samples from the plurality of subjects. In one or more examples of this disclosure, a sample of the plurality of samples comprises a tissue biopsy sample, a liquid biopsy sample, or a normal control. In one or more examples of this disclosure, the sample is a liquid biopsy sample and comprises blood, plasma, cerebrospinal fluid, sputum, stool, urine, or saliva. In one or more examples of this disclosure, the sample is a liquid biopsy sample and comprises circulating tumor cells (CTCs). In one or more examples of this disclosure, the sample is a liquid biopsy sample and comprises cell-free DNA (cfDNA), circulating tumor DNA (ctDNA), or any combination thereof.

In one or more examples of this disclosure, the plurality of nucleic acid molecules comprises a mixture of tumor nucleic acid molecules and non-tumor nucleic acid molecules. In one or more examples of the embodiment described above, the tumor nucleic acid molecules are derived from a tumor portion of a heterogeneous tissue biopsy sample, and the non-tumor nucleic acid molecules are derived from a normal portion of the heterogeneous tissue biopsy sample. In one or more examples of this disclosure, a sample of the plurality of samples comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In one or more examples of this disclosure, the one or more adapters comprise amplification primers, flow cell adaptor sequences, substrate adapter sequences, or sample index sequences. In one or more examples of this disclosure, the captured nucleic acid molecules are captured from the amplified nucleic acid molecules by hybridization to one or more bait molecules. In one or more examples of this disclosure, the one or more bait molecules comprise one or more nucleic acid molecules, each comprising a region that is complementary to a region of a captured nucleic acid molecule.

In one or more examples of this disclosure, amplifying nucleic acid molecules comprises performing a polymerase chain reaction (PCR) amplification technique, a non-PCR amplification technique, or an isothermal amplification technique. In one or more examples of this disclosure, the sequencing comprises use of a massively parallel sequencing (MPS) technique, whole genome sequencing (WGS), whole exome sequencing, targeted sequencing, direct sequencing, or Sanger sequencing technique. In such embodiments, the sequencing comprises massively parallel sequencing, and the massively parallel sequencing technique comprises next generation sequencing (NGS).

In one or more examples of this disclosure, the sequencer comprises a next generation sequencer. In one or more examples of this disclosure, one or more of the plurality of sequencing reads overlap one or more gene loci within one or more subgenomic intervals in a sample of the plurality of samples. In such embodiments the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

In one or more examples of this disclosure, the one or more gene loci comprise ABL1, ACVR1B, AKT1, AKT2, AKT3, ALK, ALOX12B, AMER1, APC, AR, ARAF, ARFRP1, ARIDIA, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKNIA, CDKNIB, CDKN2A, CDKN2B, CDKN2C, CEBPA, CHEK1, CHEK2, CIC, CREBBP, CRKL, CSFIR, CSF3R, CTCF, CTNNA1, CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1. HGF, HNF1A, HRAS, HSD3B1, ID3, IDH1, IDH2, IGFIR, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MSTIR, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2RIA, PPP2R2A, PRDM1, PRKARIA, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSCIL1, WT1, XPO1, XRCC2, ZNF217, ZNF703, or any combination thereof.

In one or more examples of this disclosure, the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.

In one or more examples of this disclosure, the method further comprises generating, by the one or more processors, a report indicating the primary site of the disease in the individual. In such embodiments, the method further comprises transmitting the report to a healthcare provider. In such embodiments, the report is transmitted via a computer network or a peer-to-peer connection.

Embodiments of the present disclosure further comprise methods for determining whether a disease in an individual is associated with a cutaneous primary site. The methods can comprise; receiving, using one or more processors, sequence read data associated with a sample from the individual, selecting, using the one or more processors, a plurality of reads from the sequence read data, determining, using the one or more processors, an ultra-violet (UV) signature metric based on the selected plurality of reads, inputting, using the one or more processors, the UV signature metric into a statistical model, and predicting, using the one or more processors, the primary site of the disease in the individual based on an output of the statistical model.

In one or more examples of this disclosure, the output is indicative of a cutaneous primary site of the disease or a non-cutaneous primary site of the disease. In one or more embodiments, the disease is cancer.

In one or more examples of this disclosure, the UV signature metric comprises a binary value indicative of a UV signature call and a confidence score associated with the UV signature call. In one or more examples of this disclosure, determining the binary value and confidence score is based on a fit of a predetermined number of short variants to a UV reference signature. In one or more examples of this disclosure, the predetermined number of short variants corresponds to three or more variants. In one or more embodiments, the UV signature metric is associated with a catalogue of somatic mutations in one or more of cancer (COSMIC) single base substitution (SBS) signature 7a, signature 7b, signature 7c, signature 7d, or a combination thereof. In one or more examples of this disclosure, the one or more mutational signatures comprise a COSMIC doublet base substitution (DBS) signature 1.

In one or more examples of this disclosure, the method further comprises determining, using the one or more processors, whether a UV signature associated with the UV signature metric was detected in the sample. In one or more examples of this disclosure, the method further comprises determining, using the one or more processors, genomic features of the selected plurality of reads, biomarker features of the selected plurality of reads, or a combination thereof. In such examples, the method further comprises inputting the genomic features of the selected plurality of reads, biomarker features of the selected plurality of reads, or a combination thereof into the statistical model to predict the primary site.

In one or more examples of this disclosure, the genomic features comprise the presence of one or more predetermined short variants or an absence of the one or more predetermined short variants. In one or more examples of this disclosure, the one or more predetermined short variants comprise a BRAF alteration, a NF1 alteration, a NRAS alteration, a NOTCH1 alteration, a NOTCH2 alteration, a NOTCH3 alteration, a PTEN alteration, a PIK3CA alteration, a PTCH1 alteration, a SMO alteration, a SUFU alteration, a TERT alteration, a TP53 alteration, a CDKN2A alteration, a RB alteration, a HRAS alteration, a KRAS alteration, a KIT alteration, a GNAQ alteration, a SF3B1 alteration, a RAC1 alteration, a MAP2K1 alteration, a MAP2K2 alteration, a CDK4 alteration, a PDGFRA alteration, a MITF alteration, a EWSR1 alteration, a STK11 alteration, a KEAP1 alteration, or a combination thereof.

In one or more examples of this disclosure, the biomarker features comprise a tumor mutational burden (TMB), one or more mutational signatures, a microsatellite instability (MSI) status, or a combination thereof. In one or more examples of this disclosure, the method further comprises receiving, at the one or more processors, clinical data. In one or more examples of this disclosure, the clinical data comprises an age of the individual, a sex of the individual, a sample type, a biopsy site of the sample, a clinicopathologic diagnosis, an immunophenotype, or a combination thereof.

In one or more examples of this disclosure, the method further comprises predicting, using the one or more processors, a type of the disease in the individual based on an output of the statistical model. In such examples, the type of the disease comprises at least one of a melanoma, a squamous cell carcinoma, a basal cell carcinoma, a pleomorphic dermal sarcoma, clear cell sarcoma, a Merkel cell carcinoma, an unspecified cutaneous carcinoma, a malignant peripheral nerve sheath tumor, or an angiosarcoma.

In one or more examples of this disclosure, the method further comprises training the statistical model. In such embodiments training the statistical model comprises receiving, using the one or more processors, training data based on a plurality of training samples and training, using the one or more processors, the statistical model based on the training data to obtain a trained statistical model. In one or more examples of this disclosure, training the statistical model comprises inputting, using the one or more processors, the training data into the statistical model, determining, using the one or more processors, a score based on the training data, and updating, using the one or more processors, one or more weights associated with the statistical model based on the score. In one or more examples of this disclosure, the training data corresponds to a plurality of training samples and comprises: one or more UV signature metrics, one or more genomic features, one or more biomarker features, one or more clinical features, or a combination thereof. In one or more examples of this disclosure, the training data further comprises a primary site associated with a respective training sample of the plurality of training samples.

In one or more examples of this disclosure, the statistical model is a machine learning model. In one or more examples of this disclosure, the statistical model is part of a machine learning process. In one or more examples of this disclosure, the statistical model includes an artificial intelligence learning model. In one or more examples of this disclosure, the statistical model comprises a classifier model. In one or more examples of this disclosure, the statistical model comprises a random forest model. In one or more examples of this disclosure, the statistical model comprises at least one of an extreme gradient boosting model, a logistic regression model, an elastic net model, a ridge regression model, a random forest model, a support vector machine model, a k-nearest neighbor model, a Bayesian model, a naïve-based model, a Gaussian naïve-based model, a clustering model, a limited expectation maximization model, a gradient boosting ensemble model, an adaboost model, a bagging model, a neural network model, a backpropagation model, a stochastic gradient descent model, a linear regression model, a non-linear regression model, a multivariate regression model, a robust machine learning model, and a proportional hazards model.

In one or more examples of this disclosure, the sequence read data for the individual is based on one or more of a broad panel sequencing, a whole exome, or a whole genome sequencing. In one or more examples of this disclosure, the sample comprises a tissue sample or a liquid sample. In one or more examples of this disclosure, the sequence read data for the individual is derived from a single biopsy sample. In one or more examples of this disclosure, the sequence read data for the individual is derived from multiple biopsy samples. In one or more examples of this disclosure, the sequence read data for the individual is derived from single cell sequencing.

In one or more examples of this disclosure, the method further comprises determining, using the one or more processors, a diagnosis for the individual based on the primary site of the disease. In one or more examples of this disclosure, the method further comprises determining, using the one or more processors, one or more of identifying a mis-diagnosis for the individual based on the primary site of the disease or refining the diagnosis of the individual. In one or more examples of this disclosure, the method further comprises assigning, using the one or more processors, a therapy for the individual based on the primary site of the disease. In one or more examples of this disclosure, the therapy comprises immune checkpoint inhibitors.

In one or more examples of this disclosure, the method further comprises determining, using the one or more processors, a stage of disease for the individual based on the primary site of the disease. In one or more examples of this disclosure, the method further comprises determining, using the one or more processors, a treatment decision for the individual based on the primary site of the disease. In one or more examples of this disclosure, the method further comprises administering, using the one or more processors, a treatment to the individual based on the primary site of the disease. In one or more examples of this disclosure, the method further comprises determining, using the one or more processors, a prognosis of the individual based on the primary site of the disease. In one or more examples of this disclosure, the method further comprises generating, using the one or more processors, a report based on the primary site of the disease. In one or more examples of this disclosure, the method further comprises determining, using the one or more processors, an eligibility of the individual for a clinical trial based on the primary site of the disease.

Embodiments of the present disclosure further include methods for diagnosing a disease. Methods for diagnosing a disease comprise diagnosing that a subject has the disease based on a determination of whether a disease is associated with a cutaneous primary site for a sample from the subject, wherein the primary site is determined according to the methods described above.

Embodiments of the present disclosure further include methods for selecting an anti-cancer therapy. Such methods include responsive to determining whether a disease for a sample from a subject is associated with a cutaneous primary site, selecting an anti-cancer therapy for the subject, wherein the primary site is determined according to the methods described above.

Embodiments of the present disclosure further include methods of treating a cancer in a subject. Such methods include responsive to determining whether a disease for a sample from a subject is associated with a cutaneous primary site, administering an effective amount of an anti-cancer therapy to the subject, wherein the primary site is determined according to the methods described above.

Embodiments of the present disclosure further include methods of monitoring cancer progression or recurrence in a subject. Such methods include determining whether a disease for a first sample from a subject is associated with a cutaneous primary site at a first time point according to the methods described above, determining whether a disease for a second sample from the subject is associated with the cutaneous primary site at a second time point, and comparing the first determination to the second determination, thereby monitoring the cancer progression or recurrence. In such embodiments, the second determination of whether the disease for the second sample from the subject is associated with the cutaneous primary site is determined according to the methods described above.

In one or more embodiments the methods for monitoring cancer progression further comprises selecting an anti-cancer therapy for the subject in response to the cancer progression. In one or more embodiments, the method further comprises administering an anti-cancer therapy to the subject in response to the cancer progression. In one or more embodiments, the method further comprises adjusting an anti-cancer therapy for the subject in response to the cancer progression. In one or more embodiments, the method further comprises adjusting a dosage of the anti-cancer therapy or selecting a different anti-cancer therapy in response to the cancer progression. In such embodiments, the method further comprises further comprising administering the adjusted anti-cancer therapy to the subject.

In one or more embodiments, the first time point is before the subject has been administered an anti-cancer therapy, and wherein the second time point is after the subject has been administered the anti-cancer therapy.

In one or more embodiments, the subject has a cancer, is at risk of having a cancer, is being routine tested for cancer, or is suspected of having a cancer. In one or more embodiments, the cancer is a solid tumor. In one or more embodiments, the cancer is a hematological cancer. In one or more embodiments, the anti-cancer therapy comprises chemotherapy, radiation therapy, immunotherapy, a targeted therapy, or surgery.

Embodiments of the present disclosure further comprise determining, identifying, or applying a cutaneous primary site for the sample as a diagnostic indicator associated with the sample. Embodiments of the present disclosure further comprise generating a genomic profile for the subject based on the determination of a cutaneous primary site of disease. In some examples, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof. In some examples, the genomic profile for the subject further comprises results from a nucleic acid sequencing-based test. In some examples, the method further comprises selecting an anti-cancer therapy, administering an anti-cancer therapy, or applying an anti-cancer therapy to the subject based on the generated genomic profile.

In one or more examples, the methods described above can be used to determine whether a disease for a sample from a subject is associated with a cutaneous primary site is used in making suggested treatment decisions for the subject. In one or more examples, the methods described above can be used to determine whether a disease for a sample from a subject is associated with a cutaneous primary site is used in applying or administering a treatment to the subject.

Systems in accordance with embodiments of the present disclosure may include: one or more processors and a memory communicatively coupled to the one or more processors. The memory may be configured to store instructions that, when executed by the one or more processors, cause the system to perform a method comprising: receiving, using one or more processors, sequence read data associated with a sample from the individual, selecting, using the one or more processors, a plurality of reads from the sequence read data, determining, using the one or more processors, an ultra-violet (UV) signature metric based on the selected plurality of reads, inputting, using the one or more processors, the UV signature metric into a statistical model, and predicting, using the one or more processors, the primary site of the disease in the individual based on an output of the statistical model.

Embodiments of the present disclosure further provide non-transitory computer-readable storage mediums. Non-transitory computer-readable storage mediums can be configured to store one or more programs, the one or more programs comprising instructions, which when executed by one or more processors of a system, cause the system to: receive, using one or more processors, sequence read data associated with a sample from the individual, select, using the one or more processors, a plurality of reads from the sequence read data, determine, using the one or more processors, an ultra-violet (UV) signature metric based on the selected plurality of reads, inputting, using the one or more processors, the UV signature metric into a statistical model, and predict, using the one or more processors, the primary site of the disease in the individual based on an output of the statistical model.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1A illustrates a non-limiting example of a process for predicting a primary site of a disease according to embodiments of the present disclosure.

FIG. 1B illustrates a non-limiting example of a process for predicting a primary site of a disease according to embodiments of the present disclosure.

FIG. 2A illustrates a non-limiting example of input features for a statistical model according to embodiments of the present disclosure.

FIG. 2B illustrates a non-limiting example of input features for a statistical model according to embodiments of the present disclosure.

FIG. 3A illustrates a non-limiting example of a process for predicting a primary site of a disease according to embodiments of the present disclosure.

FIG. 3B illustrates a non-limiting example of a process for predicting a primary site of a disease according to embodiments of the present disclosure.

FIG. 4 illustrates a non-limiting example of a process for training a statistical model according to embodiments of the present disclosure.

FIG. 5A illustrates a non-limiting example of a process for training a statistical model according to embodiments of the present disclosure.

FIG. 5B illustrates a non-limiting example of a process for training a statistical model according to embodiments of the present disclosure.

FIG. 6 illustrates a non-limiting example of a chart that illustrates the presence of various genomic features associated with samples from a plurality of individuals according to embodiments of the present disclosure.

FIG. 7 illustrates an exemplary computing device or system in accordance with one or more embodiments of the present disclosure.

FIG. 8 illustrates an exemplary computer system or computer network, in accordance with some instances of the systems described herein.

FIG. 9 illustrates non-limiting examples of histopathological samples, in accordance with some instances of the systems described herein.

### DETAILED DESCRIPTION

Disclosed herein are methods and systems for determining a primary site of a disease in an individual. For example, one or more embodiments of the present disclosure may relate to using one or more UV signatures (e.g., COSMIC UV mutational signatures) to predict the primary site of disease. In one or more examples, embodiments of the present disclosure may be well suited to determining whether the primary site is cutaneous in origin. For example, COSMIC single base substitution signature 7 is associated with UV-mediated mutagenesis and may be suggestive of a cutaneous disease origin when identified in tumor samples. Accordingly, embodiments of the present disclosure relate to systems and methods that leverage the presence or absence of a UV signature to predict the primary site and/or diagnosis. In one or more embodiments, a UV signature metric indicative of the presence or absence of the UV signature in the sample may be used in conjunction with the genomic profile (i.e., the presence or absence of genomic alterations), tumor mutational burden (TMB) and/or other features to predict the primary site and/or diagnosis. In some instances, embodiments of the present disclosure may be used to predict the primary site of disease for a sample previously determined to be a cancer of an uncertain primary site.

Embodiments of the present disclosure may be further used to overturn incorrectly rendered diagnoses. Additionally, embodiments of the present disclosure may be used to correctly identify a stage of a cancer. For example, a one-centimeter tumor identified in the lungs, where the primary site of the disease corresponds to the lungs may be determined to be a stage one cancer. However, the same tumor identified in the lungs, where the primary site of the disease may correspond to a different site may, be determined to be a stage four cancer, e.g., because the disease has metastasized. Correctly staging a disease may have important prognosis and treatment implications. For example, for a stage one cancer in the lungs, removal of the tumor may be an effective treatment option, while for a stage four cancer of cutaneous origin, immunotherapy may be an effective treatment option. Further, embodiments of the present disclosure may predict the primary site and/or diagnosis of a disease using liquid biopsy samples, which are less invasive than tissue samples required for histopathological examination. Thus, embodiments of the present disclosure can address some of the limitations of conventional histopathology by providing another metric for consideration by healthcare providers when determining a diagnosis.

Embodiments of the present disclosure further comprise methods for determining whether a disease in an individual is associated with a cutaneous primary site. The methods can comprise: receiving, using one or more processors, sequence read data associated with a sample from the individual, selecting, using the one or more processors, a plurality of reads from the sequence read data, determining, using the one or more processors, an ultra-violet (UV) signature metric based on the selected plurality of reads, inputting, using the one or more processors, the UV signature metric into a statistical model, and predicting, using the one or more processors, the primary site of the disease in the individual based on an output of the statistical model.

Systems in accordance with embodiments of the present disclosure may include: one or more processors and a memory communicatively coupled to the one or more processors. The memory may be configured to store instructions that, when executed by the one or more processors, cause the system to perform a method comprising: receiving, using one or more processors, sequence read data associated with a sample from the individual, selecting, using the one or more processors, a plurality of reads from the sequence read data, determining, using the one or more processors, an ultra-violet (UV) signature metric based on the selected plurality of reads, inputting, using the one or more processors, the UV signature metric into a statistical model, and predicting, using the one or more processors, the primary site of the disease in the individual based on an output of the statistical model.

Embodiments of the present disclosure further provide non-transitory computer-readable storage mediums. Non-transitory computer-readable storage mediums can be configured to store one or more programs, the one or more programs comprising instructions, which when executed by one or more processors of a system, cause the system to: receive, using one or more processors, sequence read data associated with a sample from the individual, select, using the one or more processors, a plurality of reads from the sequence read data, determine, using the one or more processors, an ultra-violet (UV) signature metric based on the selected plurality of reads, inputting, using the one or more processors, the UV signature metric into a statistical model, and predict, using the one or more processors, the primary site of the disease in the individual based on an output of the statistical model.

In some examples of this disclosure, the output is indicative of a cutaneous primary site of the disease or a non-cutaneous primary site of the disease. In some examples of this disclosure, the UV signature metric comprises a binary value indicative of a UV signature call and a confidence score associated with the UV signature call. In some examples of this disclosure, determining the binary value and confidence score is based on a fit of a predetermined number of short variants to a UV reference signature. In some examples of this disclosure, the predetermined number of short variants corresponds to three or more variants.

In some examples of this disclosure, the UV signature metric is associated with a catalogue of somatic mutations in one or more of cancer (COSMIC) single base substitution (SBS) signature 7a, signature 7b, signature 7c, signature 7d, or a combination thereof. In some examples, the one or more mutational signatures comprise a COSMIC doublet base substitution (DBS) signature 1. In some examples of this disclosure, the method further comprises determining, using the one or more processors, whether a UV signature corresponding to the UV signature metric was detected in the sample.

In some examples of this disclosure, the method further comprises determining, using the one or more processors, genomic features of the selected plurality of reads, biomarker features of the selected plurality of reads, or a combination thereof. In some examples, the method further comprises inputting the genomic features of the selected plurality of reads, biomarker features of the selected plurality of reads, or a combination thereof into the statistical model to predict the primary site.

In some examples of this disclosure, the method further comprises determining, using the one or more processors, genomic features of the selected plurality of reads, biomarker features of the selected plurality of reads, or a combination thereof. In some examples, the method further comprises inputting the genomic features of the selected plurality of reads, biomarker features of the selected plurality of reads, or a combination thereof into the statistical model to predict the primary site. In some examples, the genomic features comprise the presence of one or more predetermined short variants or an absence of the one or more predetermined short variants.

Accordingly, embodiments of the present disclosure provide systems and methods that can predict the primary site of disease based on one or more input features including the presence or absence of a UV signature. In one or more embodiments, a UV signature metric (e.g., indicative of the presence or absence of the UV signature) may be used in conjunction with the genomic profile (i.e., the presence or absence of genomic alterations), tumor mutational burden (TMB) and/or other features to predict the primary site and/or diagnosis. In some instances, embodiments of the present disclosure may be used to predict the primary site of disease for a sample previously determined to be a cancer of an uncertain primary site. Embodiments of the present disclosure may be further used to overturn incorrectly rendered diagnoses. Additionally, embodiments of the present disclosure may be used to correctly identify a stage of a cancer. Additionally, embodiments of the present disclosure may utilize liquid biopsy samples to determine the primary site of disease. Thus, embodiments of the present disclosure can address some of the limitations of conventional histopathology by providing another metric for consideration by healthcare providers when determining a diagnosis.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

As used herein, the term “primary site” may refer to the part of the body (e.g., organ) where the disease originated.

The section headings used herein are for organizational purposes only and are not to be construed as limiting the subject matter described.

### Methods for Determining a Primary Site of a Disease

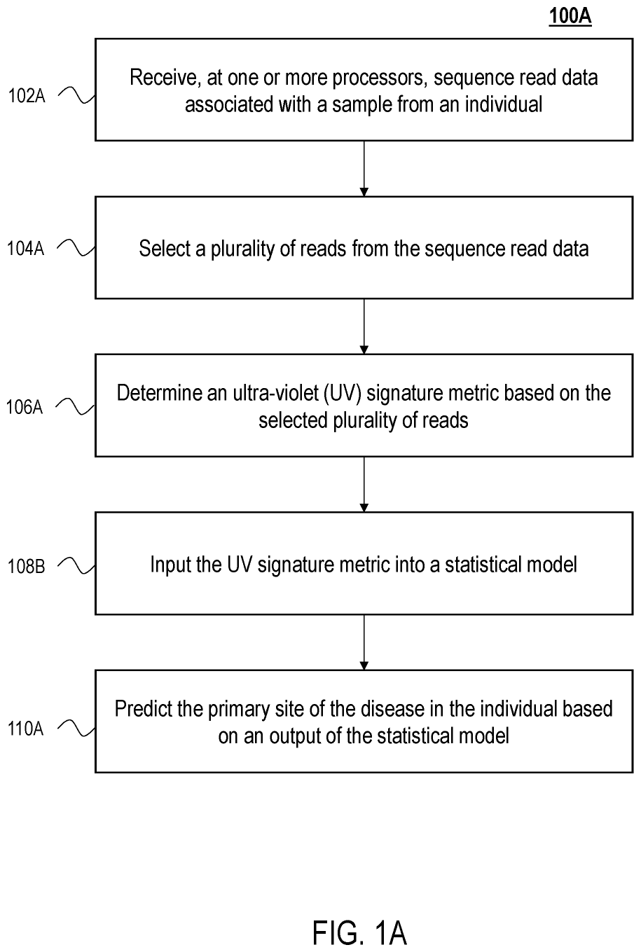

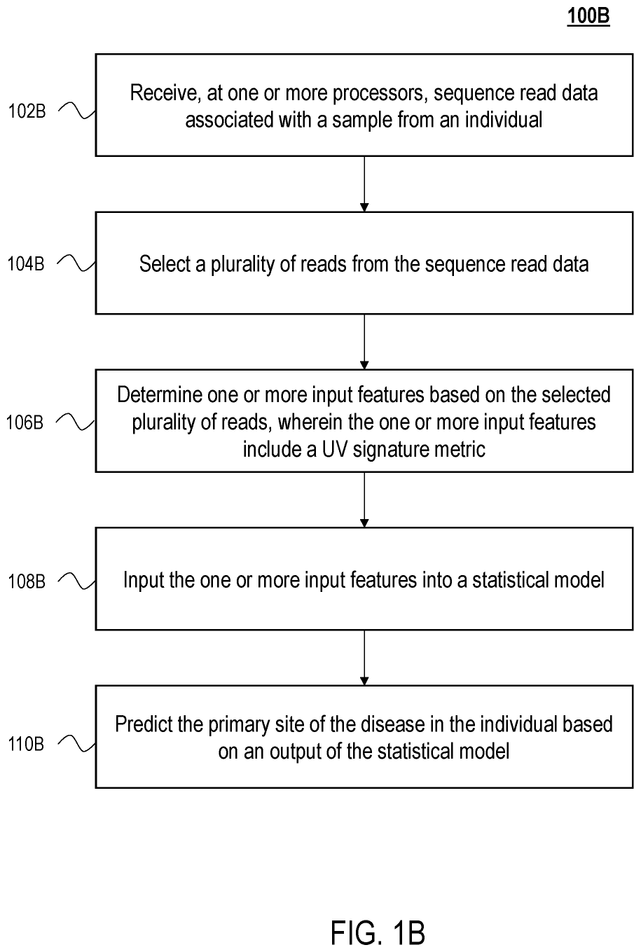

FIG. 1A provides a non-limiting example of a process 100A for predicting a primary site of a disease based on a sample from an individual. As shown in the figure, the system may predict the primary site of the disease based on a determination of a UV signature metric indicative of the presence or absence of a UV signature in the sample. Process 100A can be performed, for example, using one or more electronic devices implementing a software platform. In some examples, process 100A is performed using a client-server system, and the blocks of process 100A are divided up in any manner between the server and a client device. In other examples, the blocks of process 100A are divided up between the server and multiple client devices. Thus, while portions of process 100A are described herein as being performed by particular devices of a client-server system, it will be appreciated that process 100A is not so limited. In other examples, process 100A is performed using only a client device or only multiple client devices. In process 100A, some blocks are, optionally, combined, the order of some blocks is, optionally, changed, and some blocks are, optionally, omitted. In some examples, additional steps may be performed in combination with the process 100A. Accordingly, the operations as illustrated (and described in greater detail below) are exemplary by nature and, as such, should not be viewed as limiting.

At block 102A of FIG. 1A, the system can receive sequence read data associated with one or more genomic variants in a sample from an individual. In one or more examples, the sample may be a solid biopsy sample or a liquid biopsy sample. In some instances, the sequence read data may be derived from single region sequencing (e.g., sequencing of a single tissue biopsy sample collected from the tumor of the individual). In some instances, the genomic data comprising sequence read data may be derived from multi-region sequencing (e.g., sequencing of multiple tissue biopsy samples collected from the tumor of the individual). In some instances, the genomic data comprising sequence read data may be derived from single cell sequencing data as opposed to bulk tumor sequencing. In some instances, the genomic data comprising sequence read data may be derived from sequencing the circulating tumor DNA in a liquid biopsy sample.

In some instances, the genomic data comprising sequence read data may be derived from targeted sequencing, e.g., targeted exome sequencing. In some instances, the genomic data comprising sequence read data may be derived from broad panel sequencing. While broad panel sequencing covers a narrower segment of the genome than whole genome sequencing, embodiments of the present disclosure may nonetheless accurately identify a UV signature. In some instances, the genomic data comprising sequence read data may be derived from, e.g., whole genome or whole exome sequencing, as opposed to targeted exome sequencing or broad panel sequencing to increase the number of genomic features (e.g., the number of short variants) detected. In one or more examples, the sequence read data may be received by the system as a BAM file.

In one or more examples, the sequence read data may be indicative of a presence or absence of one or more short variants (SVs) in a patient sample. In one or more examples, the sequence read data may also be indicative of the presence or absence of genomic events, such as copy number alterations, rearrangements, insertions, deletions, fusions, chromosomal aneuploidy, whole genome doubling, Catalogue Of Somatic Mutations In Cancer (COSMIC) mutational signatures, microsatellite instability (MSI) status, tumor mutational burden (TMB), or any combination thereof. In one or more examples, the sequence read data can be indicative of features associated with a genomic event such as a location of the genomic event, whether the genomic event is in-strand, an orientation of the genomic event, a directionality of the genomic event, genes involved in the genomic event, and the like.

At block 104A of FIG. 1A, the system can select a plurality of reads from the sequence read data. In one or more examples, the system can select a plurality of reads based on a genomic event or feature of interest. For example, the system may select a plurality of reads that are associated with one or more COSMIC mutational signatures, such as ultra-violet (UV) signatures. A skilled artisan will understand that the features of interest discussed above are not limiting and the plurality of reads can be selected based on an association with additional features.

At block 106A of FIG. 1A, the system can determine one or more input features, e.g., a UV signature metric, based on the selected plurality of reads. In one or more examples, the system can determine one or more UV signature metrics by detecting variants associated with a UV signature based on the selected plurality of reads. For example, the system can identify one or more genomic variants in the sample to build a mutational profile and compare the mutational profile to one or more UV reference signatures to determine a fit of the selected plurality of reads to the one or more UV reference signature. The UV signature metric may be generated based on the determined fit and may be indicative of whether a UV signature is detected.

In one or more examples, the input feature corresponding to the UV signature metric may include a UV signature value indicative of a UV signature call (e.g., indicative of whether the UV signature is present or absent in the sample) and a UV signature confidence score (e.g., indicative of the likely accuracy of the UV signature call). In one or more examples, the UV signature value may include a numeric value or a binary value. In one or more examples, the confidence score may correspond to a numeric value or a categorical value. The UV signature value may be determined based on a fit of a number of predetermined genomic variants (e.g., short variants) to a UV reference signature as described above. In one or more examples, the UV reference signature may include at least five genomic variants. In some examples, the UV reference signature may include three or four genomic variants. Methods in accordance with the present disclosure may use a small number of genomic variants because the selected variants are used in a specific context for determining the UV signature, e.g., for detecting missense and dinucleotide changes. However, more genomic variants may be included in the UV reference signature without departing from the scope of this disclosure. In one or more examples, a greater number of variants may be associated with a higher confidence score.

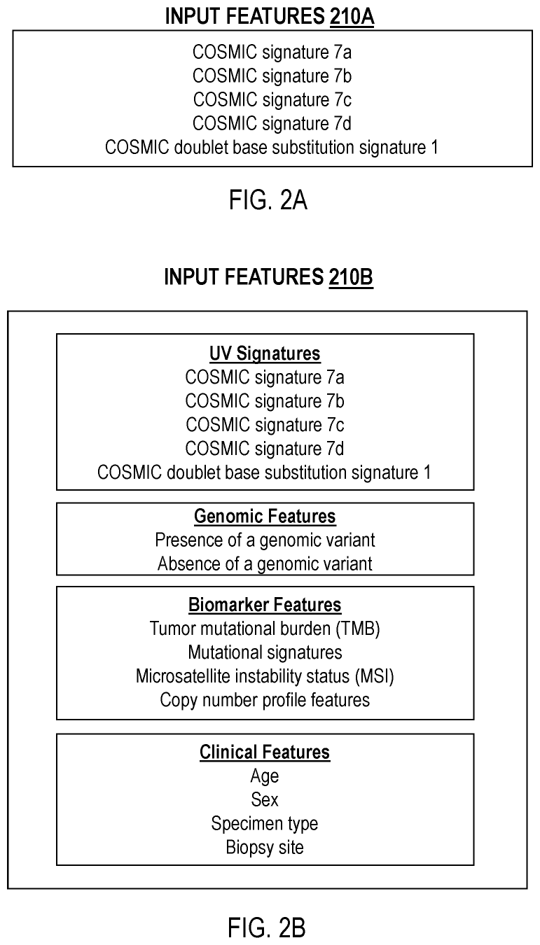

In one or more examples, the one or more input features, e.g., one or more UV signature metrics, may be associated with a COSMIC mutational signature. FIG. 2A illustrates exemplary types of input features 210A that may be determined at block 106A, according to one or more embodiments of this disclosure. For example, referring to FIG. 2A, the system can determine input features 210A associated with, but not limited to, a COSMIC signature 7a, a COSMIC signature 7b, a COSMIC signature 7c, a COSMIC signature 7d, and/or a COSMIC doublet base substitution (DBS) signature 1. In one or more examples, the input feature (e.g., UV signature metric) can correspond to a presence or absence of a mutational signature (e.g., COSMIC signature) and a confidence value indicative of the accuracy of the determined presence or absence. To the extent that input features 210A include multiple features, e.g., multiple COSMIC signatures, a skilled artisan will understand that more or less features may be determined without departing from the scope of this disclosure.

At block 108A of FIG. 1A, the system can input the one or more input features, e.g., one or more UV signature metrics, into a statistical model configured to predict a primary site of a disease of an individual based on the one or more input features. For example, the system may use the model to predict whether the disease associated with a sample has a cutaneous origin. In one or more examples, the statistical model can be a trained machine learning model. In one or more examples, the trained machine learning model may be a classifier model, for example, a random forest model.

In one or more examples, the statistical model may be part of a machine learning process. In one or more examples, the machine learning model can include an artificial intelligence (“AI”) learning model. In some instances, the machine learning model can be at least one of a supervised model or an unsupervised model. In one or more examples, the machine learning model can include one or more machine learning models, such as an extreme gradient boosting model, a logistic regression model, an elastic net model, a ridge regression model, a random forest model, a support vector machine model, a k-nearest neighbor model, a Bayesian model, a naïve-based model, a Gaussian naïve-based model, a clustering model, a limited expectation maximization model, a gradient boosting ensemble model, an adaboost model, a bagging model, a neural network model, a backpropagation model, a stochastic gradient descent model, a linear regression model, a non-linear regression model, a multivariate regression model, a robust machine learning model, and a proportional hazards model.

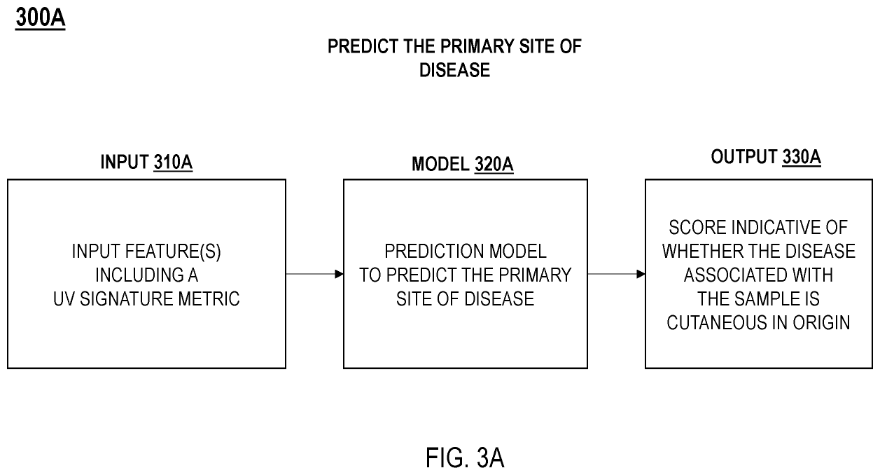

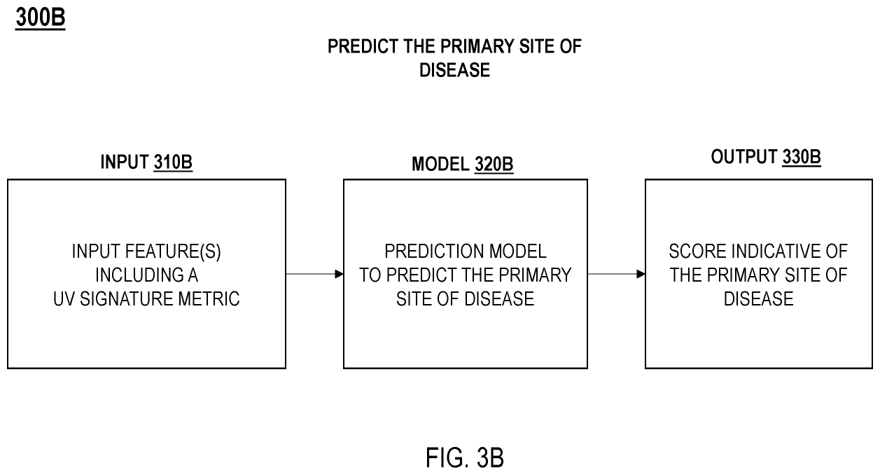

FIG. 3A illustrates an exemplary statistical model in accordance with one or more embodiments of the present disclosure. As shown in the figure, input 310A, corresponding to one or more input features (e.g., input features 210A) associated with a sample from an individual having a disease, can be input into model 320A. In one or more examples, the input features can be associated with a UV signature. The model 320A can be a prediction model configured to predict whether the disease is cutaneous or non-cutaneous in origin. The model 320A can be configured to output 330A a score indicative of whether the disease associated with the sample is cutaneous in origin.

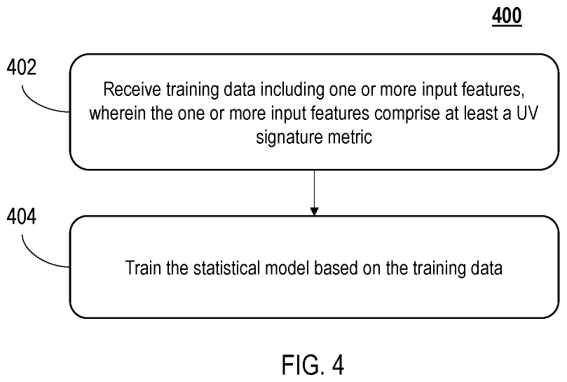

In one or more examples, the model 320A may correspond to a trained statistical model. FIG. 4 illustrates an exemplary process 400 for training a statistical model according to embodiments of this disclosure (e.g., model 320A). Process 400 can be performed, for example, using one or more electronic devices implementing a software platform. In some examples, process 400 is performed using a client-server system, and the blocks of process 400 are divided up in any manner between the server and a client device. In other examples, the blocks of process 400 are divided up between the server and multiple client devices. Thus, while portions of process 400 are described herein as being performed by particular devices of a client-server system, it will be appreciated that process 400 is not so limited. In other examples, process 400 is performed using only a client device or only multiple client devices. In process 400, some blocks are, optionally, combined, the order of some blocks is, optionally, changed, and some blocks are, optionally, omitted. In some examples, additional steps may be performed in combination with the process 400. Accordingly, the operations as illustrated (and described in greater detail below) are exemplary by nature and, as such, should not be viewed as limiting.

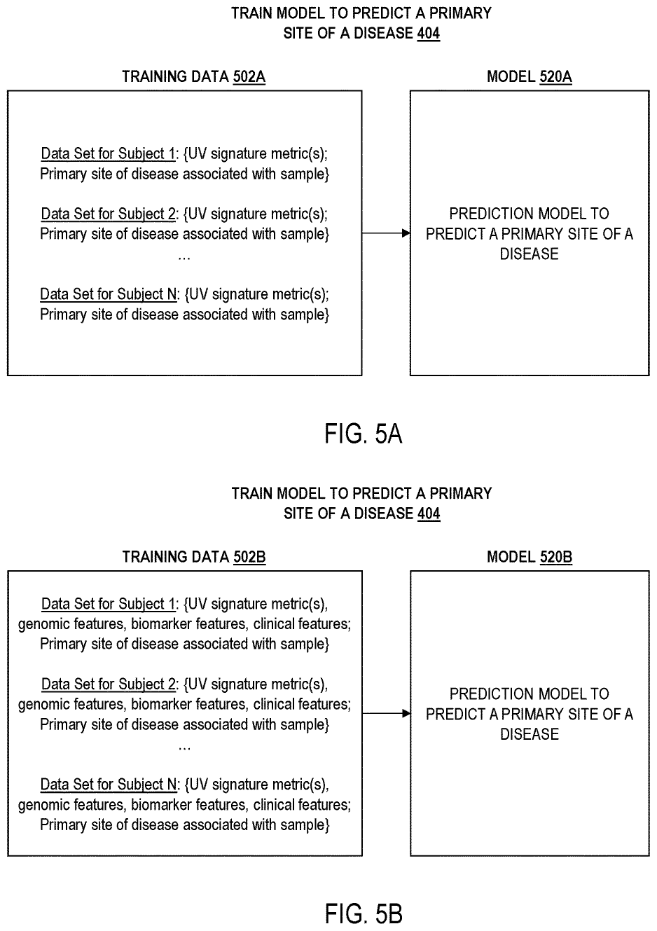

At block 402 of FIG. 4, the system may receive training data including one or more input features. In some instances, the system may receive one or more training input features including input features corresponding to one or more UV signature metrics. FIG. 5A provides an exemplary schematic showing a training process including training data 502A and an untrained model 520A. As shown in the figure, the training data 502A may include input features corresponding to one or more UV signature metrics, as well as a primary site of disease associated with a sample. In one or more examples, the model may be an unsupervised training model and the primary site of disease associated with the sample may be omitted.

At block 404 of FIG. 4, the system can train the model (e.g., model 320A and/or 520A) using the training data to obtain a trained statistical model. In one or more examples, training the statistical model may comprise an iterative process that includes inputting the training data (e.g., training data 502A) into a statistical model (e.g., statistical model 520A) and determining a score based on the training data. In one or more examples, one or more weights associated with the statistical model may be iteratively updated based on the score. In one or more examples, the model may be trained and validated using an 80/20 split of the training data set, where 80% of the training data is used to train the model, while 20% of the data is used to test and validate the model. In some instances, other training data splits, e.g., 90/10, 70/30, 60/40, may be used without departing from the scope of this disclosure.

Returning to FIG. 1A, at block 110A, the system can predict the primary site of the disease in the individual based on an output of the statistical model. In one or more examples, the system may determine whether the primary site is cutaneous or non-cutaneous in origin. For example, if the UV signature was detected in the sample, the system may determine that the primary site of the disease has a cutaneous origin; if the UV signature is not detected in the sample, the system may determine that the primary site of the disease is not cutaneous in origin.

FIG. 1B provides a non-limiting example of a process 100B for predicting a primary site of a disease based on a sample from an individual. As shown in the figure, the system may predict the primary site of the disease based on one or more input features. The input features associated with process 100B can include input features associated with UV signatures as well as other types of input features.

Process 100B can be performed, for example, using one or more electronic devices implementing a software platform. In some examples, process 100B is performed using a client-server system, and the blocks of process 100B are divided up in any manner between the server and a client device. In other examples, the blocks of process 100B are divided up between the server and multiple client devices. Thus, while portions of process 100B are described herein as being performed by particular devices of a client-server system, it will be appreciated that process 100B is not so limited. In other examples, process 100B is performed using only a client device or only multiple client devices. In process 100B, some blocks are, optionally, combined, the order of some blocks is, optionally, changed, and some blocks are, optionally, omitted. In some examples, additional steps may be performed in combination with the process 100B. Accordingly, the operations as illustrated (and described in greater detail below) are exemplary by nature and, as such, should not be viewed as limiting.

At block 102B of FIG. 1B, the system can receive sequence read data associated with a genomic variant in a sample from an individual. In one or more embodiments, block 102B of FIG. 1B can correspond to block 102A of FIG. 1A.

At block 104B of FIG. 1B, the system can select a plurality of reads from the sequence read data. In one or more examples, the system can select a plurality of reads based on a genomic event or feature of interest. For example, the system may select a plurality of reads that are associated with one or more COSMIC mutational signatures, e.g., ultra-violet (UV) signatures. In one or more examples, the system may select a plurality of reads associated with the presence or an absence of a genomic variant. In one or more examples, the system can select a plurality of reads associated with one or more biomarker features such as a TMB status, MSI status, copy number features, and/or the presence of one or more non-UV signatures. A skilled artisan will understand that the features discussed above are not limiting and the plurality of reads can be selected based on an association with additional features.

At block 106B of FIG. 1B, the system can determine one or more input features based on the selected plurality of reads. In one or more examples, the exemplary input features may include, but are not limited to input features associated with COSMIC mutational signatures (e.g., such as, but not limited to UV signatures), genomic features, biomarker features, and clinical features.

FIG. 2B illustrates exemplary input features 210B that may be determined by the system according to one or more embodiments of this disclosure. As shown in the figure, the types of input features may include, but are not limited to, UV signatures, genomic features, biomarker features, and clinical features.

In one or more examples, the UV signature metrics may be associated with a COSMIC signature 7a, a COSMIC signature 7b, a COSMIC signature 7c, a COSMIC signature 7d, and/or a COSMIC doublet base substitution (DBS) signature 1. As discussed above, the input feature corresponding to the UV signature metrics may include a UV signature value indicative of a UV signature call (e.g., indicative of if the UV signature is present or absent in the sample) and a UV signature confidence score (e.g., indicative of the likely accuracy of the UV signature call). The UV signature value may be determined based on a fit of a predetermined number of short variants to a UV reference signature, where a greater number of variants included in the UV reference signature may be associated with a higher confidence score.

In one or more examples, the genomic features may include a presence of a genomic variant or an absence of a genomic variant. In one or more examples, the input features corresponding to the genomic variant may include a value indicating whether a genomic variant is present or absent. In one or more examples, the genomic variants may include, but are not limited to BRAF, NF1, NRAS, NOTCH1, NOTCH2, NOTCH3, PTEN, PIK3CA, PTCH1, SMO, SUFU, TERT, TP53, CDKN2A, RB1, HRAS, KRAS, KIT, GNAQ, SF3B1, RAC1, MAP2K1, MAP2K2, CDK4, PDGFRA, MITF, EWSR1, STK11, KEAP1. In one or more examples, the biomarker features may include TMB, mutational signatures (e.g., aside from UV signatures), an MSI status, and copy number features (e.g., a copy number value, a zygosity value, etc.). In one or more examples, input features corresponding to these biomarker features may correspond to values or categorical labels indicative of the respective biomarker feature. A skilled artisan will understand that the input features 210B shown in FIG. 2B are exemplary and more or less features may be included without departing from the scope of this disclosure.

In one or more examples, the system can further determine one or more clinical features associated with the sample. For example, the system may receive an indication of the one or more clinical features. As shown in FIG. 2B, the one or more clinical features may include, but are not limited to, an age of the individual, a sex of the individual, a specimen type obtained from the individual (e.g., needle biopsy, tissue resection, etc.), and a biopsy site of an individual (e.g., including but not limited to lymph node, soft tissue, brain and/or the central nervous system, bone, lung, liver, colon, bladder, diaphragm, eye, pancreas, and breast). In one or more examples, input features corresponding to these clinical features may correspond to values or categorical labels indicative of the respective clinical feature.

At block 108B of FIG. 1B, the system can input the one or more input features into a statistical model. For example, the system can input the one or more input features 210B into the statistical model. The statistical model may be configured to predict a primary site of a disease of an individual based on the one or more input features. In one or more examples, the statistical model can be a trained machine learning model. In one or more examples, the trained machine learning model may be a random forest model. In one or more examples, other types of models may be used, as described above with respect to block 108A.

FIG. 3B illustrates an exemplary statistical model in accordance with one or more embodiments of the present disclosure. As shown in the figure, input 310B, corresponding to one or more input features, can be input into model 320B. Input 310B may include at least one UV signature metric. In one or more examples, the input features can further include values or categorical labels associated with UV signatures, genomic features, biomarker features, and clinical features, as described above with respect to FIG. 2B. The model 320B can be a prediction model to predict the primary site of disease. The model 320B can be configured to output 330B a score indicative of the primary site of disease.

In one or more examples, the model 320B may correspond to a trained model. In one or more examples, model 320B may be trained using process 400 as described below.

At block 402 of FIG. 4, the system may receive training data including one or more input features. In some instances, the system may receive one or more training input features including input features corresponding to one or more UV signature metrics as well as one or more other types of input features. FIG. 5B provides an exemplary schematic showing training data 502B being input into model 520B. As shown in the figure, the training data 502B may include input features corresponding to one or more UV signature metrics, one or more genomic features, one or more biomarker features, one or more clinical features, as well as a primary site of disease associated with a sample. In one or more examples, the model may be an unsupervised training model and the primary site of disease associated with the sample may be omitted.

At block 404 of FIG. 4, the system can train the model (e.g., model 320B, 520B) using the training data (e.g., 502B) to obtain a trained statistical model. In one or more examples, training the statistical model may comprise an iterative process that includes inputting the training data (e.g., training data 502B) into a statistical model (e.g., statistical model 520B) and determining a score based on the training data. In one or more examples, one or more weights associated with the statistical model may be iteratively updated based on the score. In one or more examples, the model may be trained and validated using an 80/20 split of the training data set, where 80% of the training data is used to train the model, while 20% of the data is used to test and validate the model.

At block 110B of FIG. 1B, the system can predict the primary site of the disease in the individual based on an output of the statistical model. In some instances, the model may be configured to output a score indicative of the primary site of disease. For example, the statistical model may leverage the one or more features to determine a score indicative of the primary site of disease and/or the type of disease. In one or more examples, the statistical model may predict the type of disease afflicting the individual. For example, an elevated TMB in combination with the presence of a UV signature may be indicative of a particular type of skin cancer. In one or more examples, the type of disease may include, but is not limited to, a melanoma, a squamous cell carcinoma, a basal cell carcinoma, a pleomorphic dermal sarcoma, clear cell sarcoma, Merkel cell carcinoma, unspecified cutaneous carcinoma, malignant peripheral nerve sheath tumor, or an angiosarcoma, and the like.

In some examples, the degree of elevation in TMB can be used in conjunction with the presence or absence of a UV signature to determine whether a primary site is cutaneous. In some examples, the presence of specific genomic alterations can be used in conjunction with the UV signature. For example, the presence of BRAF, NF1, and/or NRAS alterations in conjunction with the presence of a UV mutational signature suggests melanoma. As another example, the presence of NOTCH1, NOTCH2, NOTCH3, PTEN, and/or PIK3CA alterations in conjunction with the presence of a UV mutational signature suggests squamous cell carcinoma. As another example, the presence of PTCH1, SMO, and/or SUFU alterations in conjunction with the presence of a UV mutational signature suggests basal cell carcinoma. As another example, the presence of TP53 and/or RB1 alterations in conjunction with the presence of a UV mutational signature suggests Merkel cell carcinoma. In some instances, the combination of a one or more genomic alterations and the absence of a UV signature may be suggestive of a particular cancer type. For example, the presence of an EWSR1-ATF1 and/or CREB 1 rearrangement and the absence of a UV signature is indicative of clear cell sarcoma. A skilled artisan will understand that this list is exemplary and other genomic alterations in conjunction with the presence or absence of a UV mutational signature may be suggestive of cancer types with a cutaneous origin.

In some instances, the presence of specific cytogenetic alterations could also be used. For example, the presence of a fragmented genome (e.g., multiple segmental chromosomal gains and losses, quantified as a genomic loss of heterozygosity score) alterations in conjunction with the presence of a UV mutational signature suggests could suggest a squamous cell carcinoma. In some instances, multiple chromosomal copy-number alterations involving chromosomes 1, 6, 7, 9, 10 and/or 11 alterations in conjunction with the presence of a UV mutational signature suggests could suggest melanoma.

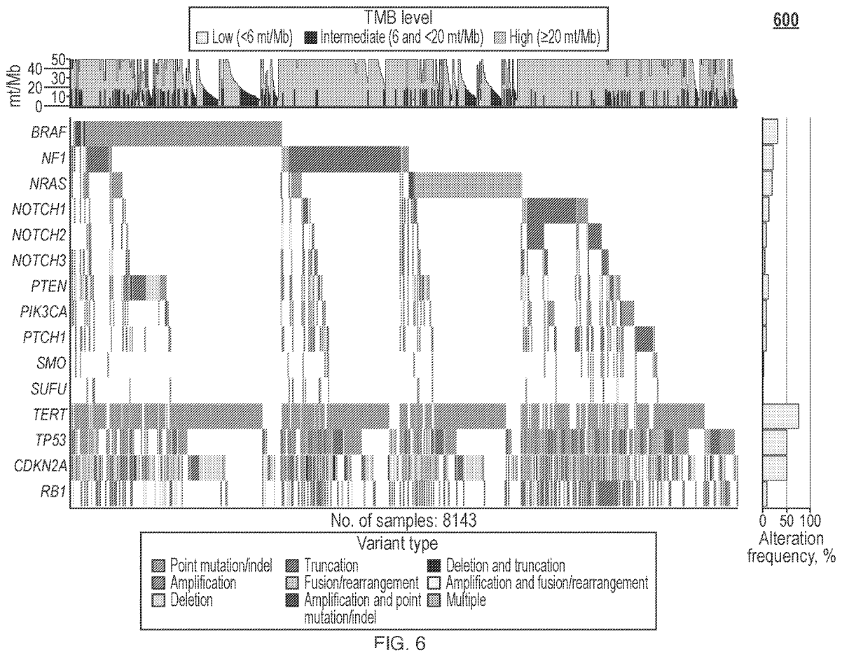

FIG. 6 illustrates a non-limiting example of a chart 900 (e.g., a tile plot) that may be associated with embodiments of the present disclosure. As shown in the figure, chart 900 illustrates the presence of various input features associated with samples from 8,143 individuals, where each sample from an individual corresponds to a single column. The TMB level, and the presence and/or absence of various genomic alterations (e.g., BRAF, NF1, NRAS, NOTCH1, NOTCH2, NOTCH3, PTEN, PIK3CA, PTCH1, SMO, SUFU, TERT, TP53, CDKN2A, RB1) for each sample is shown in chart 900. As shown in the figure, certain input features may co-occur. For example, as shown in chart 900 many individuals with a BRAF mutation also have a TERT promoter mutation. Some of these individuals with the BRAF mutation have a TP53 or CDKN2A mutation. On the other hand, BRAF mutations tend to not co-occur with SMO and SUFU mutations as well as NF1 and NRAS mutations. In one or more examples, certain diseases may be associated with the occurrence of particular mutations. For instance, BRAF alterations may be associated with melanomas, as another example, PTEN and PIK3CA alterations may be indicative of squamous cell carcinoma. In some instances, PTCH1 alterations may be associated with basal cell carcinoma; MYC amplification may be associated with angiosarcoma; and TP53 and RB1 inactivation may be associated with UV-driven Merkel cell carcinoma. Accordingly, embodiments of the present disclosure may be able to determine a type of disease based on the presence or absence of various input features, including, but not limited to the presence or absence of genomic variants, biomarker features, and/or clinical features.

In some instances, e.g., if the system determines that the disease has a cutaneous origin, the statistical model may output a type of disease, including but not limited to, a melanoma, a squamous cell carcinoma, a basal cell carcinoma, a pleomorphic dermal sarcoma, clear cell sarcoma, Merkel cell carcinoma, unspecified cutaneous carcinoma, malignant peripheral nerve sheath tumor, or an angiosarcoma, and the like. In some instances, if the system determines that the disease does not have a cutaneous origin, the output may correspond to a type of disease, including but not limited to, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, and the like.

In some instances, the disclosed methods may be used to determine a primary site of a disease, e.g., whether a disease is cutaneous in origin, by assessing one or more input features, including an input feature associated with a UV signature (e.g., UV signature metric), in at least 1, at least 2, at least 3, at least 4, at least 5, at least 6, at least 7, at least 8, at least 9, at least 10, at least 20, at least 30, at least 40, at least 50, at least 100, at least 200, at least 300, at least 400, at least 500, at least 600, at least 700, or more than 700 gene loci.

In some instances, the disclosed methods may be used to identify variants in the ABL1, ACVR1B. AKT1, AKT2, AKT3, ALK, ALOX12B, AMER1, APC, AR, ARAF, ARFRP1, ARIDIA, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKNIA, CDKNIB, CDKN2A, CDKN2B, CDKN2C, CEBPA, CHEK1, CHEK2, CIC, CREBBP, CRKL, CSFIR, CSF3R, CTCF, CTNNA1, CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGFIR, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MSTIR, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCDILG2, PDGFRA, PDGFRβ, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKARIA, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSCIL1, WT1, XPO1, XRCC2, ZNF217, or ZNF703 gene locus, or any combination thereof.

In some instances, the disclosed methods may be used to identify variants in the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene locus, or any combination thereof.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for determining a primary site of a disease may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). As used throughout this section, determining a primary site may of a disease may correspond to determining whether a disease is associated with a cutaneous primary site, e.g., is cutaneous in origin. In some instances, the disclosed methods may be applicable to diagnosis of any of a variety of cancers as described elsewhere herein.

In some instances, the disclosed methods for determining a primary site of a disease in an individual may be used to predict genetic disorders in fetal DNA. (e.g., for invasive or non-invasive prenatal testing). For example, sequence read data obtained by sequencing fetal DNA extracted from samples obtained using invasive amniocentesis, chorionic villus sampling (cVS), or fetal umbilical cord sampling techniques, or obtained using non-invasive sampling of cell-free DNA (cfDNA) samples (which comprises a mix of maternal cfDNA and fetal cfDNA), may be processed according to the disclosed methods to identify variants, e.g., copy number alterations, associated with, e.g., Down Syndrome (trisomy 21), trisomy 18, trisomy 13, and extra or missing copies of the X and Y chromosomes.

In some instances, the disclosed methods for determining a primary site of a disease may be used to select a subject (e.g., a patient) for a clinical trial based on the primary site of disease determined. In some instances, patient selection for clinical trials based on, e.g., identification of the primary site, may accelerate the development of targeted therapies and improve the healthcare outcomes for treatment decisions.

In some instances, the disclosed methods for determining a primary site of a disease may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

In some instances, the targeted therapy (or anti-cancer target therapy) may comprise abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Belcodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane 1131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligco), moxctumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the disclosed methods for determining a primary site of a disease in an individual may be used in treating a disease (e.g., a cancer) in the individual. For example, in response to determining the primary site of disease in an individual using any of the methods disclosed herein, an effective amount of an anti-cancer therapy or anti-cancer treatment may be administered to the subject.

In some instances, the disclosed methods for determining a primary site of a disease in an individual may be used for monitoring disease progression or recurrence (e.g., cancer or tumor progression or recurrence) in a subject. For example, in some instances, the methods may be used to determine a primary site of a disease in a first sample obtained from the subject at a first time point, and used to determine a primary site of a disease in a second sample obtained from the subject at a second time point, where comparison of the first determination of the primary site of the disease and the second determination of the primary site of the disease is used by a healthcare provider to monitor disease progression or recurrence. In some instances, the first time point is chosen before the subject has been administered a therapy or treatment, and the second time point is chosen after the subject has been administered the therapy or treatment.

In some instances, if a first sample from the subject was taken from a first lesion site and a second sample was taken from a second lesion of unknown origin, the methods disclosed herein could be used to compare the samples to determine if there is a clonal relationship between the tumors, e.g., to determine if the tumors have the same primary site. In some examples, a healthcare provider may use this technique monitor disease progression and can determine whether a patient has a relapse.

In some instances, a healthcare provider can monitor disease progression in a patient over time by tracking the variants that comprise the UV reference signature, e.g., where the variants are associated with the biology of cutaneous tumors.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the determination of the primary site of the disease.

In some instances, the primary site of disease determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for determining a primary site of a disease may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for determining a primary site of a disease as part of a genomic profiling process (or inclusion of the output from the disclosed methods for determining a primary site of a disease as part of the genomic profile of the subject) can improve the validity of, e.g., disease detection calls and treatment decisions, made on the basis of the genomic profile by, for example, independently confirming the primary site of disease in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo (dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other. e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pincaloma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypercosinophilic syndrome, systemic mastocytosis, familiar hypercosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMOL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde-or paraformaldehyde-fixed, paraffin-embedded (FFPE) tissues are disclosed in, e.g., Cronin, et al., (2004) Am J Pathol. 164 (1): 35-42; Masuda, et al., (1999) Nucleic Acids Res. 27 (22): 4436-4443; Specht, et al., (2001) Am J Pathol. 158 (2): 419-429; the Ambion RecoverAllTM Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.R FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4 (11): 903-5; Hodges, E. et al. (2007) Nat. Genet. 39 (12): 1522-7; and Okou, D. T. et al. (2007) Nat. Methods 4 (11): 907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 103, 104, 105 or more than 105 molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLID, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27: 455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (scc, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147 (1): 195-197), the Striped Smith-Waterman algorithm (see, e.g., Farrar (2007), “Striped Smith-Waterman Speeds Database Searches Six Times Over Other SIMD Implementations”, Bioinformatics 23 (2): 156-161), the Needleman-Wunsch algorithm (Needleman, et al. (1970) “A General Method Applicable to the Search for Similarities in the Amino Acid Sequence of Two Proteins”, J. Molecular Biology 48 (3): 443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Mutation Calling

Base calling refers to the raw output of a sequencing device, e.g., the determined sequence of nucleotides in an oligonucleotide molecule. Mutation calling refers to the process of selecting a nucleotide value, e.g., A. G. T. or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26 (5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85 (6): 847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜le-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20 (9): 1297-303; Ye. K., et al., Bioinformatics, 2009; 25 (21): 2865-71; Lunter, G., and Goodson, M., Genome Res. 2011; 21 (6): 936-9; and Li, H., et al. (2009), Bioinformatics 25 (16): 2078-9.

Methods for generating indel calls and individual-level genotype likelihoods include, e.g., the Dindel algorithm (Albers, C. A., et al., Genome Res. 2011; 21 (6): 961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21 (6): 952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 Mar. 15; 26 (6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of mutation calling methods is provided in, e.g., International Patent Application Publication No. WO 2020/236941, the entire content of which is incorporated herein by reference.

### Systems

Also disclosed herein are systems designed to implement any of the disclosed methods for predicting a primary site of disease in a sample from a subject. The systems may comprise, e.g., one or more processors, and a memory unit communicatively coupled to the one or more processors and configured to store instructions that, when executed by the one or more processors, cause the system to: receive, using one or more processors, sequence read data associated with a sample from the individual, select, using the one or more processors, a plurality of reads from the sequence read data, determine, using the one or more processors, an ultra-violet (UV) signature metric based on the selected plurality of reads, inputting, using the one or more processors, the UV signature metric into a statistical model, and predict, using the one or more processors, the primary site of the disease in the individual based on an output of the statistical model.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLID) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for predicting a primary site of disease in any of a variety of samples as described herein (e.g., a tissue sample, biopsy sample, hematological sample, or liquid biopsy sample derived from the subject).

In some instances, the plurality of gene loci for which sequencing data is processed to determine a primary site of disease may comprise at least 1, 2, 3, 4, 5, 6, 7, 8, 9, 10, 50, 100, 200, 300, 400, 500, or more than 500 gene loci.

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of a primary site of disease is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

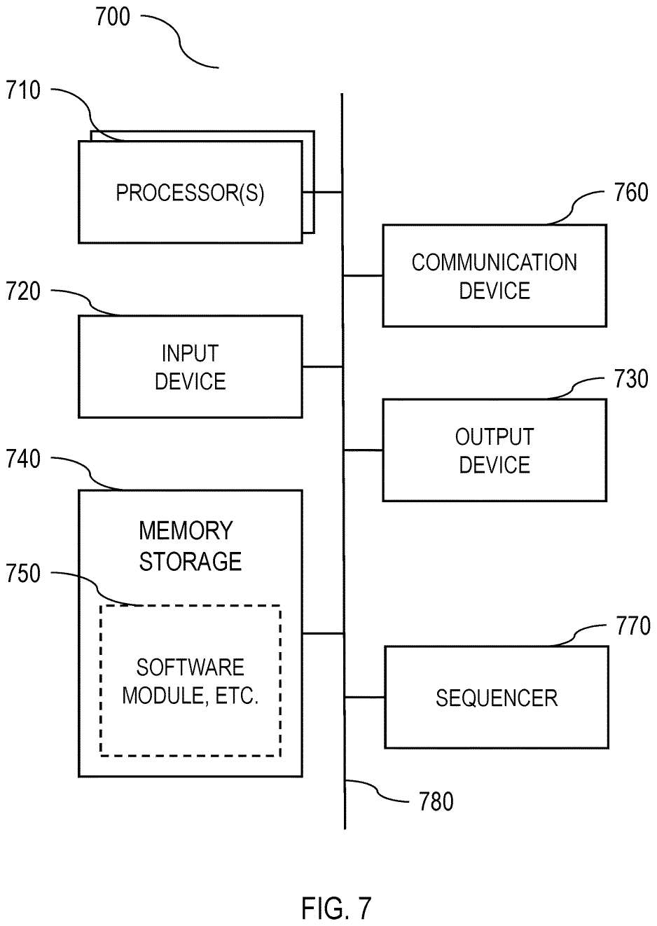

FIG. 7 illustrates an example of a computing device or system in accordance with one embodiment. Device 700 can be a host computer connected to a network. Device 700 can be a client computer or a server. As shown in FIG. 7, device 700 can be any suitable type of microprocessor-based device, such as a personal computer, workstation, server or handheld computing device (portable electronic device) such as a phone or tablet. The device can include, for example, one or more processor(s) 710, input devices 720, output devices 730, memory or storage devices 740, communication devices 760, and nucleic acid sequencers 770. Software 750 residing in memory or storage device 740 may comprise, e.g., an operating system as well as software for executing the methods described herein. Input device 720 and output device 730 can generally correspond to those described herein, and can either be connectable or integrated with the computer.

Input device 720 can be any suitable device that provides input, such as a touch screen, keyboard or keypad, mouse, or voice-recognition device. Output device 730 can be any suitable device that provides output, such as a touch screen, haptics device, or speaker.

Storage 740 can be any suitable device that provides storage (e.g., an electrical, magnetic or optical memory including a RAM (volatile and non-volatile), cache, hard drive, or removable storage disk). Communication device 760 can include any suitable device capable of transmitting and receiving signals over a network, such as a network interface chip or device. The components of the computer can be connected in any suitable manner, such as via a wired media (e.g., a physical system bus 780, Ethernet connection, or any other wire transfer technology) or wirelessly (e.g., Bluetooth®, Wi-Fi®, or any other wireless technology).

Software module 750, which can be stored as executable instructions in storage 740 and executed by processor(s) 710, can include, for example, an operating system and/or the processes that embody the functionality of the methods of the present disclosure (e.g., as embodied in the devices as described herein).

Software module 750 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 740, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 750 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 700 may be connected to a network (e.g., network 704, as shown in FIG. 7 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 700 can be implemented using any operating system, e.g., an operating system suitable for operating on the network. Software module 750 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 710.

Device 700 can further include a sequencer 770, which can be any suitable nucleic acid sequencing instrument.

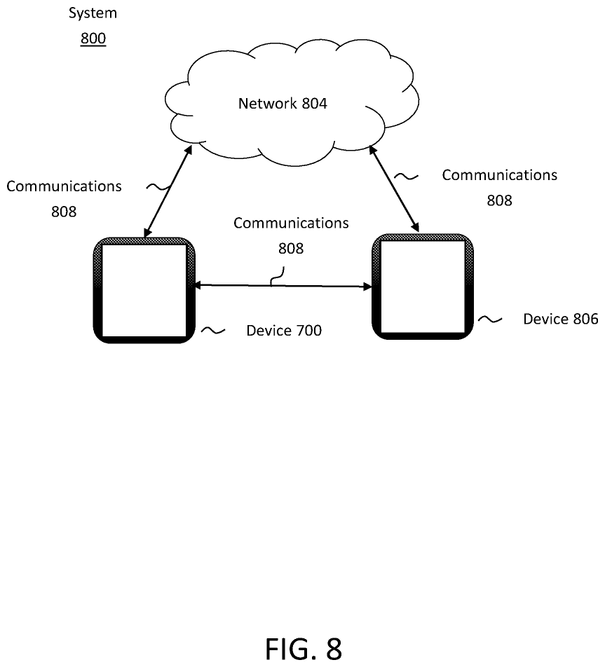

FIG. 8 illustrates an example of a computing system in accordance with one embodiment. In system 800, device 700 (e.g., as described above and illustrated in FIG. 7) is connected to network 804, which is also connected to device 806. In some embodiments, device 806 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio® RS system.

Devices 700 and 806 may communicate, e.g., using suitable communication interfaces via network 804, such as a Local Area Network (LAN), Virtual Private Network (VPN), or the Internet. In some embodiments, network 804 can be, for example, the Internet, an intranet, a virtual private network, a cloud network, a wired network, or a wireless network. Devices 700 and 806 may communicate, in part or in whole, via wireless or hardwired communications, such as Ethernet, IEEE 802.11b wireless, or the like. Additionally, devices 700 and 806 may communicate, e.g., using suitable communication interfaces, via a second network, such as a mobile/cellular network. Communication between devices 700 and 806 may further include or communicate with various servers such as a mail server, mobile server, media server, telephone server, and the like. In some embodiments, Devices 700 and 806 can communicate directly (instead of, or in addition to, communicating via network 804), e.g., via wireless or hardwired communications, such as Ethernet, IEEE 802.11b wireless, or the like. In some embodiments, devices 700 and 806 communicate via communications 808, which can be a direct connection or can occur via a network (e.g., network 804).

One or all of devices 700 and 806 generally include logic (e.g., http web server logic) or are programmed to format data, accessed from local or remote databases or other sources of data and content, for providing and/or receiving information via network 804 according to various examples described herein.

### Examples

### Example 1-Analysis of Histopathological Samples

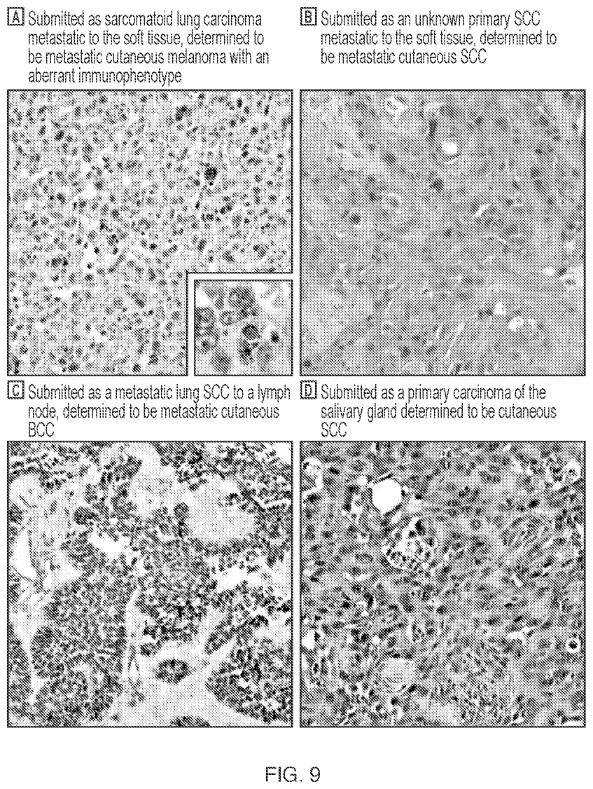

FIG. 9 illustrates non-limiting examples of histopathological samples that were presented to clinicians to determine a primary site of a disease. Sample A illustrates was determined by a clinician to be a sarcomatoid lung carcinoma metastatic to the soft tissue of the lung. Sample A was then processed according to embodiments of this disclosure and was determined to be a metastatic cutaneous melanoma with an aberrant immunophenotype. Accordingly, embodiments of the present disclosure can be used to verify and/or correct diagnoses that were primarily based on histopathological samples.

Sample B of FIG. 9, illustrates to a histopathological sample that was classified as an unknown primary squamous cell carcinoma (SCC) metastatic to the soft tissue. Sample B was then processed according to embodiments of this disclosure and was determined to be metastatic cutaneous SCC. Accordingly, embodiments of the present disclosure can be used to determine the primary site for samples that were previously associated with an unknown primary site.

Sample C of FIG. 9, illustrates to a histopathological sample that was classified as a metastatic lung SCC to a lymph node. Sample C was then processed according to embodiments of this disclosure and was determined to be a metastatic cutaneous basal cell carcinoma (BCC). Accordingly, embodiments of the present disclosure can be used to verify and/or correct diagnoses that were primarily based on histopathological samples.

Sample D of FIG. 9, illustrates to a histopathological sample that was classified as a primary carcinoma of the salivary gland. Sample D was then processed according to embodiments of this disclosure and was determined to be a cutaneous SCC. Accordingly, embodiments of the present disclosure can be used to verify and/or correct diagnoses that were primarily based on histopathological samples.

### Example 2-Accuracy of UV Mutational Signature Analysis

Table 1 provides a non-limiting example of statistics for the accuracy of using the disclosed UV mutational signature analysis methods to identify patients with cutaneous primary tumors using cell-free DNA liquid biopsy sampling techniques. In addition to considering all patient cases included in a clinical genomics database, two sensitivity analyses were performed-one using patient cases with elevated tumor fraction, the other using only patient cases with elevated tumor mutational burden (TMB) (i.e., where signature analysis was possible).

TABLE 1
Accuracy of UV mutational analysis for the identification of patients
with cutaneous primary tumors using cell-free DNA liquid biopsy.

| | | | | | Signature Analysis | |
|---|---|---|---|---|---|---|
| | All LB Cases | | cTF >10% | | Possible | |
| | (n = 19,122) | | (n = 4,749) | | (n = 2,685) | |
| Statistic | Value | 95% CI | Value | 95% CI | Value | 95% CI |
| Sensitivity | 16.8% | 12.0% to | 38.2% | 25.4% to | 60.6% | 47.8% to |
| | | 22.7% | | 52.3% | | 72.4% |
| Specificity | 99.7% | 99.6% to | 99.6% | 99.3% to | 98.1% | 97.5% to |
| | | 99.8% | | 99.7% | | 98.6% |
| Positive | 57.9 | 38.6 to | 89.6 | 51.6 to | 32.4 | 23.08 to |
| Likelihood | | 86.8 | | 155.6 | | 45.46 |
| Ratio | | | | | | |
| Negative | 0.83 | 0.78 to | 0.62 | 0.50 to | 0.4 | 0.30 to |
| Likelihood | | 0.89 | | 0.76 | | 0.54 |
| Ratio | | | | | | |
| Disease | 1.10% | 0.92% to | 1.2% | 0.9% to | 2.5% | 1.91% to |
| Prevalence | | 1.21% | | 1.5% | | 3.12% |
| (*) | | | | | | |
| Positive | 38.2% | 29.2% to | 51.2% | 37.7% to | 44.9% | 36.78% to |
| Predictive | | 48.1% | | 64.6% | | 53.39% |
| Value (*) | | | | | | |
| Negative | 99.1% | 99.1% to | 99.3% | 99.1% to | 99.0% | 98.65% to |
| Predictive | | 99.2% | | 99.4% | | 99.26% |
| Value (*) | | | | | | |
| Accuracy (*) | 98.8% | 98.7% to | 98.9% | 98.5% to | 97.2% | 96.51% to |
| | | 99.0% | | 99.1% | | 97.80% |

Sensitivity, specificity, disease prevalence, positive and negative predictive value as well as accuracy are expresses as percentages.
Confidence intervals for sensitivity, specificity, and accuracy are “exact” Clopper-Pearson confidence intervals.
Confidence intervals for the likelihood ratios are calculated using the “log method” as given on page 109 of Altman et al. (2000) Statistics with Confidence, 2nd ed. BMJ Books.
Confidence intervals for the predictive values are the standard logit confidence intervals given by Mercaldo et al. (2007), “Confidence Intervals for Predictive Values with an Emphasis to Case-Control Studies”, Stat Med. 26 (10): 2170-83, except when the predictive value is 0 or 100%, in which case a Clopper-Pearson confidence interval is reported.

### Exemplary Implementations

Exemplary implementations of the methods and systems described herein include:
1. A method for determining whether a disease in an individual is associated with a cutaneous primary site, the method comprising:
   providing a plurality of nucleic acid molecules obtained from a sample from a subject;
   ligating one or more adapters onto one or more nucleic acid molecules from the plurality of nucleic acid molecules;
   amplifying the one or more ligated nucleic acid molecules from the plurality of nucleic acid molecules;
   capturing amplified nucleic acid molecules from the amplified nucleic acid molecules;
   sequencing, by a sequencer, the captured nucleic acid molecules to obtain a plurality of sequence reads that represent the captured nucleic acid molecules;
   receiving, at one or more processors, sequence read data for the plurality of sequence reads;
   determining, using the one or more processors, an ultra-violet (UV) signature metric based on the selected plurality of reads;
   inputting, using the one or more processors, the UV signature metric into a statistical model; and
   predicting, using the one or more processors, the primary site of the disease in the individual based on an output of the statistical model.
2. The method of clause 1, wherein the output is indicative of a cutaneous primary site of the disease or a non-cutaneous primary site of the disease.
3. The method of clause 1 or clause 2, wherein the UV signature metric comprises a binary value indicative of a UV signature call and a confidence score associated with the UV signature call.
4. The method of clause 3, wherein determining the binary value and confidence score is based on a fit of a predetermined number of short variants to a UV reference signature.
5. The method of clause 4, wherein the predetermined number of short variants corresponds to three or more variants.
6. The method of any of clauses 1 to 5, wherein the UV signature metric is associated with a catalogue of somatic mutations in one or more of cancer (COSMIC) single base substitution (SBS) signature 7a, signature 7b, signature 7c, signature 7d, or a combination thereof.
7. The method of clause 6, wherein the one or more mutational signatures comprise a COSMIC doublet base substitution (DBS) signature 1.
8. The method of any of clauses 1 to 7, further comprising determining, using the one or more processors, whether a UV signature corresponding to the UV signature metric was detected in the sample.
9. The method of any of clauses 1 to 8, further comprising determining, using the one or more processors, genomic features of the selected plurality of reads, biomarker features of the selected plurality of reads, or a combination thereof.
10. The method of clause 9, further comprising inputting the genomic features of the selected plurality of reads, biomarker features of the selected plurality of reads, or a combination thereof into the statistical model to predict the primary site.
11. The method of any of clauses 1 to 10, further comprising determining, using the one or more processors, genomic features of the selected plurality of reads, biomarker features of the selected plurality of reads, or a combination thereof.
12. The method of clause 11, further comprising inputting the genomic features of the selected plurality of reads, biomarker features of the selected plurality of reads, or a combination thereof into the statistical model to predict the primary site.
13. The method of any of clauses 10 to 12, wherein the genomic features comprise the presence of one or more predetermined short variants or an absence of the one or more predetermined short variants.
14. The method of any one of clauses 1 to 13, wherein the disease is cancer.
15. The method of clause 14, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypercosinophilic syndrome, systemic mastocytosis, familiar hypercosinophilia, chronic eosinophilic leukemia, neuroendocrine cancers, or a carcinoid tumor.
16. The method of clause 15, wherein the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.
17. The method of any one of clauses 14 to 16, further comprising treating the subject with an anti-cancer therapy.
18. The method of clause 17, wherein the anti-cancer therapy comprises a targeted anti-cancer therapy.
19. The method of clause 18, wherein the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane 1131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligco), moxctumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.
20. The method of any one of clauses 1 to 19, further comprising obtaining the sample from the subject.
21. The method of any one of clauses 1 to 20, wherein the sample comprises a tissue biopsy sample, a liquid biopsy sample, or a normal control.
22. The method of clause 21, wherein the sample is a liquid biopsy sample and comprises blood, plasma, cerebrospinal fluid, sputum, stool, urine, or saliva.
23. The method of clause 21, wherein the sample is a liquid biopsy sample and comprises circulating tumor cells (CTCs).
24. The method of clause 21, wherein the sample is a liquid biopsy sample and comprises cell-free DNA (cfDNA), circulating tumor DNA (ctDNA), or any combination thereof.
25. The method of any one of clauses 1 to 24, wherein the plurality of nucleic acid molecules comprises a mixture of tumor nucleic acid molecules and non-tumor nucleic acid molecules.
26. The method of clause 25, wherein the tumor nucleic acid molecules are derived from a tumor portion of a heterogeneous tissue biopsy sample, and the non-tumor nucleic acid molecules are derived from a normal portion of the heterogeneous tissue biopsy sample.
27. The method of clause 25, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.
28. The method of any one of clauses 1 to 27, wherein the one or more adapters comprise amplification primers, flow cell adaptor sequences, substrate adapter sequences, or sample index sequences.
29. The method of any one of clauses 1 to 28, wherein the captured nucleic acid molecules are captured from the amplified nucleic acid molecules by hybridization to one or more bait molecules.
30. The method of clause 29, wherein the one or more bait molecules comprise one or more nucleic acid molecules, each comprising a region that is complementary to a region of a captured nucleic acid molecule.
31. The method of any one of clauses 1 to 30, wherein amplifying nucleic acid molecules comprises performing a polymerase chain reaction (PCR) amplification technique, a non-PCR amplification technique, or an isothermal amplification technique.
32. The method of any one of clauses 1 to 31, wherein the sequencing comprises use of a massively parallel sequencing (MPS) technique, whole genome sequencing (WGS), whole exome sequencing, targeted sequencing, direct sequencing, or Sanger sequencing technique.
33. The method of clause 32, wherein the sequencing comprises massively parallel sequencing, and the massively parallel sequencing technique comprises next generation sequencing (NGS).
34. The method of any one of clauses 1 to 33, wherein the sequencer comprises a next generation sequencer.
35. The method of any one of clauses 1 to 34, wherein one or more of the plurality of sequencing reads overlap one or more gene loci within one or more subgenomic intervals in the sample.
36. The method of clause 35, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.
37. The method of clause 35 or clause 36, wherein the one or more gene loci comprise ABL1, ACVR1B, AKT1, AKT2, AKT3, ALK, ALOX12B, AMER1, APC, AR, ARAF, ARFRP1, ARIDIA, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF. BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKNIA, CDKNIB, CDKN2A, CDKN2B, CDKN2C, CEBPA, CHEK1, CHEK2, CIC, CREBBP, CRKL, CSFIR, CSF3R, CTCF, CTNNA1, CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGFIR, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MSTIR, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCDILG2, PDGFRA, PDGFRβ, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKARIA, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSCIL1, WT1, XPO1, XRCC2, ZNF217, ZNF703, or any combination thereof.
38. The method of clause 35 or clause 36, wherein the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.
39. The method of any one of clauses 1 to 38, further comprising generating, by the one or more processors, a report indicating the primary site of the disease in the individual.
40. The method of clause 39, further comprising transmitting the report to a healthcare provider.
41. The method of clause 40, wherein the report is transmitted via a computer network or a peer-to-peer connection.
42. A method for determining whether a disease in an individual is associated with a cutaneous primary site, the method comprising:
   receiving, using one or more processors, sequence read data associated with a sample from the individual;
   selecting, using the one or more processors, a plurality of reads from the sequence read data;
   determining, using the one or more processors, an ultra-violet (UV) signature metric based on the selected plurality of reads;
   inputting, using the one or more processors, the UV signature metric into a statistical model; and
   predicting, using the one or more processors, the primary site of the disease in the individual based on an output of the statistical model.
43. The method of clause 42, wherein the output is indicative of a cutaneous primary site of the disease or a non-cutaneous primary site of the disease.
44. The method of clause 42 or clause 43, wherein the disease is cancer.
45. The method of any of clauses 42 to 44, wherein the UV signature metric comprises a binary value indicative of a UV signature call and a confidence score associated with the UV signature call.
46. The method of clause 45, wherein determining the binary value and confidence score is based on a fit of a predetermined number of short variants to a UV reference signature.
47. The method of clause 46, wherein the predetermined number of short variants corresponds to three or more variants.
48. The method of any of clauses 42 to 47, wherein the UV signature metric is associated with a catalogue of somatic mutations in one or more of cancer (COSMIC) single base substitution (SBS) signature 7a, signature 7b, signature 7c, signature 7d, or a combination thereof.
49. The method of clause 48, wherein the one or more mutational signatures comprise a COSMIC doublet base substitution (DBS) signature 1.
50. The method of any of clauses 42 to 49, further comprising determining, using the one or more processors, whether a UV signature associated with the UV signature metric was detected in the sample.
51. The method of any of clauses 42 to 50, further comprising determining, using the one or more processors, genomic features of the selected plurality of reads, biomarker features of the selected plurality of reads, or a combination thereof.
52. The method of clause 51, further comprising inputting the genomic features of the selected plurality of reads, biomarker features of the selected plurality of reads, or a combination thereof into the statistical model to predict the primary site.
53. The method of any of clauses 51 to 52, wherein the genomic features comprise the presence of one or more predetermined short variants or an absence of the one or more predetermined short variants.
54. The method of clause 53, wherein the one or more predetermined short variants comprise a BRAF alteration, a NF1 alteration, a NRAS alteration, a NOTCH1 alteration, a NOTCH2 alteration, a NOTCH3 alteration, a PTEN alteration, a PIK3CA alteration, a PTCH1 alteration, a SMO alteration, a SUFU alteration, a TERT alteration, a TP53 alteration, a CDKN2A alteration, a RB alteration, a HRAS alteration, a KRAS alteration, a KIT alteration, a GNAQ alteration, a SF3B1 alteration, a RAC1 alteration, a MAP2K1 alteration, a MAP2K2 alteration, a CDK4 alteration, a PDGFRA alteration, a MITF alteration, a EWSR1 alteration, a STK11 alteration, a KEAP1 alteration, or a combination thereof.
55. The method of any of clauses 51 to 54, wherein the biomarker features comprise a tumor mutational burden (TMB), one or more mutational signatures, a microsatellite instability (MSI) status, or a combination thereof.
56. The method of any of clauses 51 to 55, further comprising receiving, at the one or more processors, clinical data.
57. The method of clause 56, wherein the clinical data comprises an age of the individual, a sex of the individual, a sample type, a biopsy site of the sample, a clinicopathologic diagnosis, an immunophenotype, or a combination thereof.
58. The method of any of clauses 51 to 57, further comprising predicting, using the one or more processors, a type of the disease in the individual based on an output of the statistical model.
59. The method of clause 58, wherein the type of the disease comprises at least one of a melanoma, a squamous cell carcinoma, a basal cell carcinoma, a pleomorphic dermal sarcoma, clear cell sarcoma, a Merkel cell carcinoma, an unspecified cutaneous carcinoma, a malignant peripheral nerve sheath tumor, or an angiosarcoma.
60. The method of clauses 42 to 59, further comprising training the statistical model, wherein training the statistical model comprises:
   receiving, using the one or more processors, training data based on a plurality of training samples; and
   training, using the one or more processors, the statistical model based on the training data to obtain a trained statistical model.
61. The method of clause 60, further comprising training the statistical model, wherein training the statistical model comprises:
   inputting, using the one or more processors, the training data into the statistical model;
   determining, using the one or more processors, a score based on the training data; and
   updating, using the one or more processors, one or more weights associated with the statistical model based on the score.
62. The method of clause 60, wherein the training data corresponds to a plurality of training samples and comprises: one or more UV signature metrics, one or more genomic features, one or more biomarker features, one or more clinical features, or a combination thereof.
63. The method of clause 62, wherein the training data further comprises a primary site associated with a respective training sample of the plurality of training samples.
64. The method of any of clauses 42 to 63, wherein the statistical model is a machine learning model.
65. The method of any of clauses 42 to 64, wherein the statistical model is part of a machine learning process.
66. The method of any of clauses 42 to 65, wherein the statistical model includes an artificial intelligence learning model.
67. The method of any of clauses 42 to 66, wherein the statistical model comprises a classifier model.
68. The method of any of clauses 42 to 67, wherein the statistical model comprises a random forest model.
69. The method of any of clauses 42 to 68, wherein the statistical model comprises at least one of an extreme gradient boosting model, a logistic regression model, an elastic net model, a ridge regression model, a random forest model, a support vector machine model, a k-nearest neighbor model, a Bayesian model, a naïve-based model, a Gaussian naïve-based model, a clustering model, a limited expectation maximization model, a gradient boosting ensemble model, an adaboost model, a bagging model, a neural network model, a backpropagation model, a stochastic gradient descent model, a linear regression model, a non-linear regression model, a multivariate regression model, a robust machine learning model, and a proportional hazards model.
70. The method any of clauses 42 to 69, wherein the sequence read data for the individual is based on one or more of a broad panel sequencing, a whole exome, or a whole genome sequencing.
71. The method of any of clauses 42 to 70, wherein the sample comprises a tissue sample or a liquid sample.
72. The method any of clauses 42 to 71, wherein the sequence read data for the individual is derived from a single biopsy sample.
73. The method any of clauses 42 to 71, wherein the sequence read data for the individual is derived from multiple biopsy samples.
74. The method any of clauses 42 to 73, wherein the sequence read data for the individual is derived from single cell sequencing.
75. The method any of clauses 42 to 74, further comprising determining, using the one or more processors, a diagnosis for the individual based on the primary site of the disease.
76. The method clause 75, further comprising determining, using the one or more processors, one or more of identifying a mis-diagnosis for the individual based on the primary site of the disease or refining the diagnosis of the individual.
77. The method any of clauses 42 to 76, further comprising assigning, using the one or more processors, a therapy for the individual based on the primary site of the disease.
78. The method of clause 77, wherein the therapy comprises immune checkpoint inhibitors.
79. The method any of clauses 42 to 78, further comprising determining, using the one or more processors, a stage of disease for the individual based on the primary site of the disease.
80. The method any of clauses 42 to 79, further comprising determining, using the one or more processors, a treatment decision for the individual based on the primary site of the disease.
81. The method any of clauses 42 to 80, further comprising administering, using the one or more processors, a treatment to the individual based on the primary site of the disease.
82. The method any of clauses 42 to 81, further comprising determining, using the one or more processors, a prognosis of the individual based on the primary site of the disease.
83. The method any of clauses 42 to 82, further comprising generating, using the one or more processors, a report based on the primary site of the disease.
84. The method any of clauses 42 to 83, further comprising determining, using the one or more processors, an eligibility of the individual for a clinical trial based on the primary site of the disease.
85. A method for diagnosing a disease, the method comprising: diagnosing that a subject has the disease based on a determination of whether a disease is associated with a cutaneous primary site for a sample from the subject, wherein the primary site is determined according to the method of any one of clauses 42 to 84.
86. A method of selecting an anti-cancer therapy, the method comprising: responsive to determining whether a disease for a sample from a subject is associated with a cutaneous primary site, selecting an anti-cancer therapy for the subject, wherein the primary site is determined according to the method of any one of clauses 42 to 84.
87. A method of treating a cancer in a subject, comprising: responsive to determining whether a disease for a sample from a subject is associated with a cutaneous primary site, administering an effective amount of an anti-cancer therapy to the subject, wherein the primary site is determined according to the method of any one of clauses 42 to 84.
88. A method for monitoring cancer progression or recurrence in a subject, the method comprising:
   determining whether a disease for a first sample from a subject is associated with a cutaneous primary site at a first time point according to the method of any one of clauses 42 to 84;
   determining whether a disease for a second sample from the subject is associated with the cutaneous primary site at a second time point; and comparing the first determination to the second determination, thereby monitoring the cancer progression or recurrence.
89. The method of clause 88, wherein the second determination of whether the disease for the second sample from the subject is associated with the cutaneous primary site is determined according to the method of any one of clauses 42 to 84.
90. The method of clause 88 or clause 89, further comprising selecting an anti-cancer therapy for the subject in response to the cancer progression.
91. The method of clause 88 or clause 89, further comprising administering an anti-cancer therapy to the subject in response to the cancer progression.
92. The method of clause 88 or clause 89, further comprising adjusting an anti-cancer therapy for the subject in response to the cancer progression.
93. The method of any one of clauses 90-92, further comprising adjusting a dosage of the anti-cancer therapy or selecting a different anti-cancer therapy in response to the cancer progression.
94. The method of clause 93, further comprising administering the adjusted anti-cancer therapy to the subject.
95. The method of any one of clauses 88 to 94, wherein the first time point is before the subject has been administered an anti-cancer therapy, and wherein the second time point is after the subject has been administered the anti-cancer therapy.
96. The method of any one of clauses 88 to 95, wherein the subject has a cancer, is at risk of having a cancer, is being routine tested for cancer, or is suspected of having a cancer.
97. The method of any one of clauses 88 to 96, wherein the cancer is a solid tumor.
98. The method of any one of clauses 88 to 96, wherein the cancer is a hematological cancer.
99. The method of any one of clauses 88 to 98, wherein the anti-cancer therapy comprises chemotherapy, radiation therapy, immunotherapy, a targeted therapy, or surgery.
100. The method of any one of clauses 42 to 84, further comprising determining, identifying, or applying a cutaneous primary site for the sample as a diagnostic indicator associated with the sample.
101. The method of any one of clauses 42 to 84, further comprising generating a genomic profile for the subject based on the determination of a cutaneous primary site of disease.
102. The method of clause 101, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.
103. The method of clause 101 or clause 102, wherein the genomic profile for the subject further comprises results from a nucleic acid sequencing-based test.
104. The method of any one of clauses 101 to 103, further comprising selecting an anti-cancer therapy, administering an anti-cancer therapy, or applying an anti-cancer therapy to the subject based on the generated genomic profile.
105. The method of any one of clauses 42 to 84, wherein the determination of whether a disease for a sample from a subject is associated with a cutaneous primary site is used in making suggested treatment decisions for the subject.
106. The method of any one of clauses 42 to 84, wherein the determination of whether a disease for a sample from a subject is associated with a cutaneous primary site is used in applying or administering a treatment to the subject.
107. A system comprising:
   one or more processors; and
   a memory communicatively coupled to the one or more processors and configured to store instructions that, when executed by the one or more processors, cause the system to perform a method comprising:
      receiving, using one or more processors, sequence read data associated with a sample from the individual;
      selecting, using the one or more processors, a plurality of reads from the sequence read data;
      determining, using the one or more processors, an ultra-violet (UV) signature based on the selected plurality of reads;
      inputting, using the one or more processors, the UV signature metric into a statistical model; and
      predicting, using the one or more processors, the primary site of the disease in the individual based on an output of the statistical model.
108. A non-transitory computer-readable storage medium storing one or more programs, the one or more programs comprising instructions, which when executed by one or more processors of a system, cause the system to perform a method comprising:
   receiving, using one or more processors, sequence read data associated with a sample from the individual;
   selecting, using the one or more processors, a plurality of reads from the sequence read data;
   determining, using the one or more processors, an ultra-violet (UV) signature based on the selected plurality of reads;
   inputting, using the one or more processors, the UV signature metric into a statistical model; and
   predicting, using the one or more processors, the primary site of the disease in the individual based on an output of the statistical model.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method for determining whether a cancer in an individual is associated with a cutaneous primary site, the method comprising:
providing a plurality of nucleic acid molecules obtained from a sample from a subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules;
receiving, at one or more processors, sequence read data for the plurality of sequence reads;
determining, using the one or more processors, an ultra-violet (UV) signature metric based on the selected plurality of reads;
inputting, using the one or more processors, the UV signature metric into a statistical model; and
predicting, using the one or more processors, the primary site of the cancer in the individual based on an output of the statistical model.

2. The method of claim 1, wherein the output is indicative of a cutaneous primary site of the cancer or a non-cutaneous primary site of the cancer.

3. (canceled)

4. The method of claim 1, wherein the UV signature metric comprises a binary value indicative of a UV signature call and a confidence score associated with the UV signature call.

5. The method of claim 4, wherein determining the binary value and confidence score is based on a fit of a predetermined number of short variants to a UV reference signature.

6. The method of claim 1, wherein the UV signature metric is associated with a catalogue of somatic mutations in one or more mutational signatures comprising a COSMIC single base substitution (SBS) signature 7a, signature 7b, signature 7c, signature 7d, or a combination thereof.

7. The method of claim 6, wherein the one or more mutational signatures comprise a COSMIC doublet base substitution (DBS) signature 1.

8. The method of claim 1, further comprising determining, using the one or more processors, whether a UV signature associated with the UV signature metric was detected in the sample.

9. The method of claim 1, further comprising determining, using the one or more processors, genomic features of the selected plurality of reads, biomarker features of the selected plurality of reads, or a combination thereof.

10. The method of claim 9, further comprising inputting the genomic features of the selected plurality of reads, biomarker features of the selected plurality of reads, or a combination thereof into the statistical model to predict the primary site.

11. The method of claim 9, wherein the genomic features comprise the presence of one or more predetermined short variants or an absence of the one or more predetermined short variants.

12. (canceled)

13. The method of claim 9, wherein the biomarker features comprise a tumor mutational burden (TMB), one or more mutational signatures, a microsatellite instability (MSI) status, or a combination thereof.

14. The method of claim 9, further comprising receiving, at the one or more processors, clinical data.

15. The method of claim 9, further comprising predicting, using the one or more processors, a type of the cancer in the individual based on an output of the statistical model.

16. The method of claim 15, wherein the type of the cancer comprises at least one of a melanoma, a squamous cell carcinoma, a basal cell carcinoma, a pleomorphic dermal sarcoma, clear cell sarcoma, a Merkel cell carcinoma, an unspecified cutaneous carcinoma, a malignant peripheral nerve sheath tumor, or an angiosarcoma.

17. The method of claim 1, further comprising training the statistical model, wherein training the statistical model comprises:
receiving, using the one or more processors, training data based on a plurality of training samples and comprising one or more UV signature metrics, one or more genomic features, one or more biomarker features, one or more clinical features, or a combination thereof; and
training, using the one or more processors, the statistical model based on the training data to obtain a trained statistical model.

18. (canceled)

19. (canceled)

20. (canceled)

21. The method of claim 1, wherein the statistical model comprises a classifier model or a random forest model.

22. (canceled)

23. The method of claim 1, wherein the sequence read data for the individual is based on one or more of a broad panel sequencing, a whole exome sequencing, a whole genome sequencing, or a single cell sequencing.

24. (canceled)

25. The method of claim 1, further comprising determining a diagnosis for the individual, assigning a therapy for the individual, determining a prognosis of the individual, or determining an eligibility of the individual for a clinical trial based on the primary site of the cancer.

26. (canceled)

27. The method of claim 25, wherein the therapy comprises immune checkpoint inhibitors.

28. (canceled)

29. (canceled)

30. A system comprising:
one or more processors; and
a memory communicatively coupled to the one or more processors and configured to store instructions that, when executed by the one or more processors, cause the system to perform a method comprising:
receiving, using one or more processors, sequence read data associated with a sample from an individual diagnosed with a cancer;
selecting, using the one or more processors, a plurality of reads from the sequence read data;
determining, using the one or more processors, an ultra-violet (UV) signature based on the selected plurality of reads;
inputting, using the one or more processors, the UV signature metric into a statistical model; and
predicting, using the one or more processors, the primary site of the cancer in the individual based on an output of the statistical model.
